SEQUENCE LISTING (1 sequence)

SEQ ID NO: 1 - protein, 16 aa, synthetic construct
GKKCGPQGIWGQCKKG

(19) United States
(12) Patent Application Publication
(10) Pub. No.: US 2026/0345555 A1
(43) Pub. Date: Nov. 5, 2026

(54) Compositions and Methods for Protecting Salivary Glands from Off-Target Radiation Damage

(71) Applicant: University of Rochester, Rochester, NY (US)

(72) Inventors: Lisa DeLouise, Rochester, NY (US); Lindsay Piraino, Rochester, NY (US); Chiao Yun Chen, Rochester, NY (US); Jared Mereness, Rochester, NY (US); Catherine Ovitt, Rochester, NY (US); Danielle Benoit, Rochester, NY (US)

(21) Appl. No.: 19/111,543

(22) PCT Filed: Sep. 15, 2023
(86) PCT No.: PCT/US2023/074359

§ 371 Date: Mar. 13, 2025

Related U.S. Application Data
(60) Provisional application No. 63/375,932, filed on Sep. 16, 2022.

(51) Int. Cl. A61K 31/4152; A61K 31/05; A61K 31/407; A61K 31/496; A61K 31/4965; A61K 31/517; A61K 45/06; A61P 43/00

(52) U.S. Cl. CPC A61K 31/4152; A61K 31/05; A61K 31/407; A61K 31/496; A61K 31/4965; A61K 31/517; A61K 45/06; A61P 43/00

(57) ABSTRACT

Provided are compositions and methods for protecting tissue of patients undergoing radiation therapy from off-target radiation damage.

### CROSS-REFERENCE TO RELATED APPLICATIONS

This Application claims priority to, and benefit of, U.S. Provisional Patent Application No. 63/375,932, filed on Sep. 16, 2022, the disclosure of which is hereby incorporated by reference in its entirety.

### STATEMENT OF GOVERNMENT SUPPORT

This invention was made with government support under DE027695 and DE002212 awarded by the National Institutes of Health. The government has certain rights in the invention.

### REFERENCE TO A “SEQUENCE LISTING” SUBMITTED AS AN XML FILE

The present application hereby incorporates by reference the entire contents of the XML file named “204606-0159-00WO_SequenceListing.xml” which was created on Sep. 15, 2023 and is 2,019 bytes in size.

### BACKGROUND OF THE INVENTION

Radiation damage of the salivary gland during head and neck cancer treatment often causes permanent loss of secretory function and reduced salivary flow. Due to the decrease in saliva production, patients have issues with eating, speaking, and swallowing. Additionally, patients are at an increased risk of oral infections and tooth decay, and hence suffer a reduced quality of life. Current treatment options, including sialogogues, mouthwashes, and chewing gum, only provide temporary relief and there is no cure (Escobar A et al., IntechOpen, 2019, pp. 15-37). Several strategies have been proposed to alleviate this damage, including cell/hydrogel transplantation, stem cell replacement, and gene therapy (Shubin A D et al., Tissue Eng: Part A, 2015, 21: 1733-1751; Shubin A D et al., Acta Biomater, 2017, 50: 437-449; Pringle S et al., Stem Cells, 2016, 34: 640-652; Baum B J et al., Oral Oncol, 2010, 46: 4-8). Despite promising results, these methods have remained experimental. A preferable approach would be to utilize preventative therapies that preserve salivary gland function. Currently intensity modulation radiation therapy (IMRT) and the radioprotective drug amifostine, an antioxidant, are used clinically to prevent salivary gland damage. IMRT involves using 3D imaging to target the radiation beams away from sensitive organs such as the salivary gland (Wang X et al., J Radiat Res, 2016, 57: i69-i75). While this method can be beneficial in some cases, there are mixed results on patient-reported claims of dry mouth and it cannot be implemented in some cases due to tumor location (Wang X et al., J Radiat Res, 2016, 57: i69-i75; Eisbruch A, J Clin Oncol, 2007, 25: 4863-4864). Amifostine is the only FDA-approved drug to prevent radiation-induced xerostomia, but due to severe side effects, including nausea, vomiting, and hypotension, it is often necessary to discontinue its use (Rades D et al., RadiotherOncol, 2004, 70: 261-264; Eisbruch A, J Clin Oncol, 2011, 29: 119-121). These drawbacks highlight the critical need for new radioprotective drugs to prevent xerostomia. The present invention addresses this unmet need.

### SUMMARY OF THE INVENTION

In one aspect, the present disclosure provides compositions and methods of preventing radiation damage to tissue. In some embodiments, the tissue being protected is any tissue(s) in the proximity of or surrounding a site receiving radiation therapy. For example, in certain embodiments, the tissue being protected comprise salivary gland tissue. In one embodiment, the method comprises administering to a subject a composition comprising one or more radioprotective compound. In one embodiment, the method comprises administering to a subject a composition comprising one or more selected from the group consisting of glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, analogs, derivatives, and prodrugs thereof, and pharmaceutically acceptable salts and hydrates thereof.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1, comprising FIG. 1A through FIG. 1D, depicts an overview of the salivary gland tissue chip. FIG. 1A depicts primary salivary gland cell clusters 20-100 m seeded into the MB arrays. FIG. 1B depicts the MB arrays. The inset is an image showing the cross-sectional view of an MB. FIG. 1C depicts clusters aggregating to form spheres within the MBs at day 7. FIG. 1D depicts a schematic representation of hydrogel encapsulation of salivary gland cell clusters within an MB chip. The schematic was made using Biorender.com (AD25LJLLID).

FIG. 2, comprising FIG. 2A through FIG. 2K, depicts glutathione levels indicating a dose-response to radioprotective drug, WR1065 and other reported radioprotective compounds. FIG. 2A depicts the timeline of drug treatment and assay for glutathione at 4 days post-radiation using Biorender.com. FIG. 2B depicts representative images of the glutathione assay for 0 Gy. FIG. 2C depicts representative images of the glutathione assay for 15 Gy. FIG. 2D depicts representative images of the glutathione assay for 15 Gy+4 mM WR1065 (WR). FIG. 2E depicts the quantification of the fluorescence intensity of individual MBs normalized to 0 Gy. N (# of chips) ≥3, n (# of MBs) ≥150. Scale bar is 600 μm. SGm tissue chips are cultured for 4 Days then exposed to WR1065 (the active form of amifostine) for 30 min prior to, during, and 30 min after radiation. Glutathione is analyzed on day 9. Using the same irradiation and drug exposure protocol, FIG. 2F depicts representative quantification of glutathione for SGms treated with Tempol and FIG. 2G depicts representative quantification of glutathione for SGms treated with Edaravone. FIG. 2H depicts representative quantification of glutathione for SGms treated with N-acetylcysteine (NAC). FIG. 2I depicts representative quantification of glutathione for SGms treated with 50 μM Rapamycin. FIG. 2J depicts representative quantification of glutathione for SGms treated with 100 ng/mL Palifermin. FIG. 2K depicts representative quantification of glutathione for SGms treated with 50 μM Ex-Rad. For FIG. 2F through FIG. 2K: N (# of chips) ≥3, n (# of MBs) ≥120. For FIG. 2E through FIG. 2K: ns, nonsignificant; compared to 15 Gy: ****, p<0.0001; compared to 0 Gy: $$$$, p<0.0001; $$$, p<0.001; $$, p<0.01. NAC, N-acetylcysteine;

FIG. 3, comprising FIG. 3A through FIG. 3K, depicts that senescence is increased with radiation and restored to unirradiated control levels with WR-1065 and other reported radioprotective compounds. FIG. 3A depicts the timeline of drug treatment and assay for senescence at 5 days post-radiation created using Biorender.com. FIG. 3B depicts representative images of the senescence assay for 0 Gy. FIG. 3C depicts representative images of the senescence assay for 15 Gy. FIG. 3D depicts representative images of the senescence assay for 15 Gy+4 mM WR-1065 (WR). FIG. 3E depicts quantification of the fluorescence intensity of individual MBs normalized to 0 Gy. N (number of chips) ≥3; n (number of MBs) ≥120. Drugs are used to treat SGm on day 4 for 30 min prior to and 30 min after radiation and senescence is analyzed on day 8. Using the same irradiation and drug exposure protocol, FIG. 3F depicts representative quantification of senescence for SGms treated with Tempol and FIG. 3G depicts representative quantification of senescence for SGms treated with Edaravone. FIG. 3H depicts representative quantification of senescence for SGms treated with N-acetylcysteine. FIG. 3I depicts representative quantification of senescence for SGms treated with 50 M Rapamycin. FIG. 3J depicts representative quantification of senescence for SGms treated with 100 ng/mL Palifermin. FIG. 3K depicts representative quantification of senescence for SGms treated with 50 μM Ex-Rad. For FIG. 3F through FIG. 3K: N (# of chips) ≥3; n (# of MBs) ≥150. For FIG. 3E through FIG. 3K: ns=nonsignificant; compared to 15 Gy: ****, p<0.0001; compared to 0 Gy: $$$$, p<0.0001. NAC, N-acetylcysteine.

FIG. 4 depicts representative results from screening a drug library of 438 compounds using the glutathione and senescence assays which identified 25 compounds that protect against post-radiation changes. FIG. 4 depicts a graph of fluorescence intensity normalized to 0 Gy as a result of different compounds. Each circle represents the normalized fluorescence intensity for the glutathione assay for each compound. White circles are compounds that were not hits. Light grey circles are compounds that were hits with the glutathione assay only. Black circles are compounds that were hits with both assays. The thin dotted line represents 0 Gy that was used for normalization of the data. The thick dotted line represents 15 Gy untreated controls using the glutathione assay.

FIG. 5, comprising FIG. 5A through FIG. 5C, depicts the identification of potential mechanisms of action on salivary gland and similarities among identified radioprotective drugs. FIG. 5A depicts a table showing drug activity related to secretion (calcium signaling), prostaglandin production, and anti-inflammatory or antioxidant properties. FIG. 5B depicts reactome pathways significantly associated with gene expression patterns affected by treatment with 12 of the identified potential radioprotective compounds (p<0.05). E-scores (−0.5 to 0.5) associated with each pathway show predicted activity related to treatment with the identified drugs. FIG. 5C depicts a heat map showing ranks for drug-pathway interactions from most-upregulating to most-downregulating drug for each pathway.

FIG. 6, comprising FIG. 6A through FIG. 6H depicts dose-response data for lead compounds from the drug screen analyzed via glutathione levels. FIG. 6A depicts a heat map of combined dose-response results with 0 Gy (1.0) at left and 15 Gy (0.3) only on right. FIG. 6B depicts representative dose response quantification of glutathione for SGms treated with phenylbutazone. FIG. 6C depicts representative dose response quantification of glutathione for SGms treated with meropenem. FIG. 6D depicts representative dose response quantification of glutathione for SGms treated with diethylstilbestrol. FIG. 6E depicts representative dose response quantification of glutathione for SGms treated with prazosin HCl. FIG. 6F depicts representative dose response quantification of glutathione for SGms treated with enoxacin. FIG. 6G depicts representative dose response quantification of glutathione for SGms treated with glipizide. FIG. 6H depicts representative dose response quantification of glutathione for SGms treated with doripenem hydrate. The dotted lines represent 0 Gy and 15 Gy, respectively. Statistics were calculated using ANOVA with Dunnett's post-hoc test. For FIG. 6B through FIG. 6H: ns, nonsignificant; **, p<0.01; ***, p<0.001; ****, p<0.0001.

FIG. 7, comprising FIG. 7A through FIG. 7H, depicts dose-response data for lead compounds from the drug screen analyzed for senescence levels. FIG. 7A depicts a heat map of combined dose-response results with 0 Gy (1.0) at left and 15 Gy (1.3) only on right. FIG. 7B depicts representative dose response quantification of senescence for SGms treated with phenylbutazone. FIG. 7C depicts representative dose response quantification of senescence for SGms treated with meropenem. FIG. 7D depicts representative dose response quantification of senescence for SGms treated with diethylstilbestrol. FIG. 7E depicts representative dose response quantification of senescence for SGms treated with prazosin HCl. FIG. 7F depicts representative dose response quantification of senescence for SGms treated with enoxacin. FIG. 7G depicts representative dose response quantification of senescence for SGms treated with glipizide. FIG. 7H depicts representative dose response quantification of senescence for SGms treated with doripenem hydrate. The dotted lines represent 0 Gy and 15 Gy, respectively. Statistics were calculated using ANOVA with Dunnett's post-hoc test. For FIG. 7B through FIG. 7H: ns, nonsignificant; **, p<0.01; ***, p<0.001; ****, p<0.0001.

FIG. 8, comprising FIG. 8A through FIG. 8I, depicts in vivo testing of double hits for radioprotection of mouse salivary gland. FIG. 8A depicts a representative image of retroductal drug administration. FIG. 8B depicts a schematic of drug treatment, irradiation, and harvest protocol. FIG. 8C depicts representative images of γH2AX staining in mouse salivary gland tissue with no radiation exposure (0 Gy control) 48 hours post treatment with vehicle. FIG. 8D depicts representative images of γH2AX staining in mouse salivary gland tissue 48 hours post treatment with vehicle followed by 15 Gy radiation exposure. FIG. 8E depicts representative images of γH2AX staining in mouse salivary gland tissue 48 hours post treatment with WR1065 followed by 15 Gy radiation exposure. FIG. 8F depicts representative images of γH2AX staining in mouse salivary gland tissue 48 hours post treatment with phenylbutazone (PB) followed by 15 Gy radiation exposure. FIG. 8G depicts representative images of γH2AX staining in mouse salivary gland tissue 48 hours post treatment with enoxacin followed by 15 Gy radiation exposure. FIG. 8H depicts representative images of γH2AX staining in mouse salivary gland tissue 48 hours post treatment with doripenem hydrate (DH) followed by 15 Gy radiation exposure. Scale bars: 20 m. FIG. 8I depicts quantification of γH2AX foci per nucleus from all treatment groups. N=4-6; compared to 0 Gy: *, p<0.05; **, p<0.01.

FIG. 9 depicts the reduced glutathione assay showing the best signal separation at 4 days post-radiation. Fluorescence intensity is normalized to 0 Gy. Statistics compare 0 Gy to 15 Gy at each timepoint. N (# of chips)≥3, n (# of MBs) ≥120; ****=p<0.0001, ***=p<0.001; ns=nonsignificant.

FIG. 10, comprising FIG. 10A through FIG. 10B, depicts Edaravone and N-acetylcysteine showing partial radioprotection using the glutathione assays. FIG. 10A depicts Edaravone tested at 0.1 mM. FIG. 10B depicts N-acetylcysteine tested at 10 mM. For FIG. 10A and FIG. 10B: N (# of chips) ≥3, n (# of MBs) ≥200; ****=p<0.0001; NAC=N-acetylcysteine

FIG. 11, depicts the β-Galactosidase senescence assay showing the greatest signal separation at 5 days post-radiation. Senescence assay tested at 5 and 7 days. N (# of chips) ≥4, n (# of MBs) ≥180; ****, p<0.0001.

FIG. 12, comprising FIG. 12A through FIG. 12B, depicts Edaravone and N-acetylcysteine being protective against radiation-induced senescence by high concentrations. FIG. 12A depicts Edaravone tested at 100 μM. FIG. 12B depicts N-acetylcysteine tested at 10 mM. For FIG. 12A and FIG. 12B: N (# of chips)=3, n (# of MBs) ≥120; ****p<0.0001; NAC=N-acetylcysteine.

FIG. 13, comprising FIG. 13A through FIG. 13F, depicts the identification of potential drug mechanisms and similarities among identified radioprotective compounds. FIG. 13A depicts E-scores associated with each pathway showing predicted activity related to treatment with the identified compounds from canonical pathways. FIG. 13B depicts a heat map showing ranks for drug-pathway interactions from most-upregulating to most-downregulating drug for each pathway from canonical pathways. FIG. 13C depicts E-scores associated with each pathway showing predicted activity related to treatment with the identified compounds from Biocarta gene sets. FIG. 13D depicts a heat map showing ranks for drug-pathway interactions from most-upregulating to most-downregulating drug for each pathway from Biocarta gene sets. FIG. 13E depicts E-scores associated with each pathway showing predicted activity related to treatment with the identified compounds from KEGG pathways. FIG. 13F depicts a heat map showing ranks for drug-pathway interactions from most-upregulating to most-downregulating drug for each pathway from KEGG pathways.

### DETAILED DESCRIPTION OF THE INVENTION

In one aspect, the disclosure is directed to methods for the prevention of off-target radiation damage, for example protection of salivary glands or other tissue during radiation treatment for cancer. In some embodiments, the radioprotection is accomplished through the administration of a composition comprising at least one radioprotective drug. In some embodiments, the composition comprises one or more selected from the group consisting of glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, analogs, derivatives, and prodrugs thereof, and pharmaceutically acceptable salts and hydrates thereof.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

The term “prodrug” refers to compounds that differ in structure from the reference molecule, but is chemically modified by a particular cellular process to ultimately become modified to retain the essential properties of the reference molecule or become the reference molecule.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

A “therapeutic” treatment is a treatment administered to a subject who exhibits signs or symptoms of pathology disease or disorder, for the purpose of diminishing or eliminating those signs or symptoms.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a sufficient amount of an agent to provide the desired biological or physiologic result. That result may be reduction and/or alleviation of a sign, a symptom, or a cause of a disease or disorder, or any other desired alteration of a biological system. An appropriate effective amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “potency” refers to the dose needed to produce half the maximal response (ED₅₀).

As used herein, the term “efficacy” refers to the maximal effect (Emax) achieved within an assay.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances, or otherwise evaluating the values or categorization of the substance or the sample.

As used herein, “associated” refers to coincidence with the development or manifestation of a disease, condition, or phenotype. Association may be due to, but is not limited to, genes responsible for housekeeping functions, those that are part of a pathway that is involved in a specific disease, condition, or phenotype and those that indirectly contribute to the manifestation of a disease, condition or phenotype.

As used herein, the term “cancer” refers to any of various types of malignant neoplasms, most of which invade surrounding tissues, may metastasize to several sites and are likely to recur after attempted removal and to cause death of the patient unless adequately treated. As used herein, neoplasia comprises cancer. Representative cancers include, for example, squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias, including non-acute and acute leukemias, such as acute myelogenous leukemia, acute lymphocytic leukemia, acute promyelocytic leukemia (APL), acute T-cell lymphoblastic leukemia, T-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas, among others, which may be treated by one or more compounds of the present invention.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based in part on the discovery that a variety of FDA-approved drugs prevent radiation damage. Accordingly, in some embodiments the invention is directed towards preventing off-target radiation damage during radiation treatment. In some embodiments, the invention is directed to protecting any tissue(s) in the proximity of or surrounding a target of radiation therapy from radiation damage. In some embodiments, the tissue being protected are the salivary glands.

In certain embodiments, the invention is directed to the use of a composition comprising at least one selected from the group consisting of glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, analogs, derivatives, and prodrugs thereof, and pharmaceutically acceptable salts and hydrates thereof.

### Compositions

In some embodiments, the present invention provides compositions comprising a radioprotective drug for preventing radiation damage. In some embodiments, the composition prevents off-target radiation damage in a tissue proximal to a targeted area receiving radiation therapy. In some embodiments, the composition prevents off-target damage to the salivary glands from radiation therapy. Exemplary agents include, but are not limited, to small molecules, isolated nucleic acids, vectors, isolated peptides, peptide mimetics, and the like.

In one embodiment, the composition of the present invention comprises a small molecule radioprotective drug. In various embodiments, the composition comprises one or more compound selected from the group consisting of glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, analogs, derivatives, and prodrugs thereof, and pharmaceutically acceptable salts and hydrates thereof.

In one embodiment, the composition comprises one or more selected from the group consisting of glipizide, meropenem, oxcarbazepine, doripenem, phenylbutazone, enoxacin, cilostazol, triamcinolone, didanosine, prazosin HCl, indomethacin, diethylstibestrol, emtricitabine, analogs, derivatives, and prodrugs thereof, and pharmaceutically acceptable salts and hydrates thereof.

In one embodiment, the composition comprises one or more selected from the group consisting of phenylbutazone, meropenem, diethylstilbestrol, prazosin HCl, enoxacin, glipizide, doripenem hydrate, analogs, derivatives, and prodrugs thereof, and pharmaceutically acceptable salts and hydrates thereof.

In one embodiment, the composition comprises one or more selected from the group consisting of phenylbutazone, prazosin HCl, enoxacin, analogs, derivatives, and prodrugs thereof, and pharmaceutically acceptable salts and hydrates thereof.

In some embodiments, the composition comprises two or more compounds selected from the group consisting of: glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, analogs, derivatives, and prodrugs thereof, and pharmaceutically acceptable salts and hydrates thereof. In some embodiments, the composition comprises three or more compounds selected from the group consisting of: glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, analogs, derivatives, and prodrugs thereof, and pharmaceutically acceptable salts and hydrates thereof.

In some embodiments, the composition comprises two or more compounds selected from the group consisting of glipizide, meropenem, oxcarbazepine, doripenem, phenylbutazone, enoxacin, cilostazol, triamcinolone, didanosine, prazosin HCl, indomethacin, diethylstibestrol, emtricitabine, analogs, derivatives, and prodrugs thereof, and pharmaceutically acceptable salts and hydrates thereof. In some embodiments, the composition comprises three or more compounds selected from the group consisting of glipizide, meropenem, oxcarbazepine, doripenem, phenylbutazone, enoxacin, cilostazol, triamcinolone, didanosine, prazosin HCl, indomethacin, diethylstibestrol, emtricitabine, analogs, derivatives, and prodrugs thereof, and pharmaceutically acceptable salts and hydrates thereof.

In some embodiments, the composition comprises two or more compounds selected from the group consisting of phenylbutazone, meropenem, diethylstilbestrol, prazosin HCl, enoxacin, glipizide, doripenem hydrate, analogs, derivatives, and prodrugs thereof, and pharmaceutically acceptable salts and hydrates thereof. In some embodiments, the composition comprises three or more compounds selected from the group consisting of phenylbutazone, meropenem, diethylstilbestrol, prazosin HCl, enoxacin, glipizide, doripenem hydrate, analogs, derivatives, and prodrugs thereof, and pharmaceutically acceptable salts and hydrates thereof.

In some embodiments, the composition comprises two or more compounds selected from the group consisting of meropenem, diethylstibestrol, glipizide, doripenem hydrate, and pharmaceutically acceptable salts thereof. In some embodiments, the composition comprises three or more compounds selected from the group consisting of meropenem, diethylstibestrol, glipizide, doripenem hydrate, analogs, derivatives, and prodrugs thereof, and pharmaceutically acceptable salts and hydrates thereof. In some embodiments, the composition comprises meropenem, diethylstibestrol, glipizide, and doripenem hydrate, or analogs, derivatives, and prodrugs thereof, and pharmaceutically acceptable salts and hydrates thereof.

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

### Methods

In some embodiments, the disclosure provides methods of preventing radiation damage. In one embodiment, the methods prevent off-target radiation damage to a tissue proximal to the target of the radiation therapy. In some embodiments, the methods prevent off-target radiation damage to the salivary glands during radiation therapy. In one embodiment, the method comprises administering to the subject an effective amount of a composition comprising a radioprotective drug.

In some embodiments, the methods are useful in preventing radiation damage associated with any form of radiation therapy. In some embodiments, the radiation therapy is for a cancer or non-cancerous disease or disorder. For example, methods of the present invention can be used for any cancer which may be treated by targeted ionizing radiation. In some embodiments, the cancer is selected from the group consisting of anal cancer, bladder cancer, bone cancer, brain cancer, breast cancer, cervical cancer, chondrosarcoma, chordoma, colon cancer, esophageal cancer, Ewing's sarcoma, head and neck cancer, leukemia, liver cancer, lung cancer, lymphoma, meningioma, non-Hodgkin's lymphoma, osteosarcoma, pancreatic cancer, paranasal sinus cancer, prostate cancer, rectal cancer, skin cancer, soft tissue sarcomas, spinal cord tumors, spine tumors, stomach cancer, uterine cancer, vaginal cancer, and vulval cancer. In some embodiments, the cancer is head and neck cancer.

In some embodiments, the radiation therapy is for treatment of a non-cancerous disease or disorder. In some embodiments, the non-cancerous disease or disorder is selected from the group consisting of acoustic neuroma, arteriovenous malformations, Graves ophthalmopathy, keloids, orbital pseudotumor, trigeminal neuralgia, and a non-cancerous tumor.

In some embodiments, the disclosure provides methods comprising administering to the subject an effective amount of a composition comprising one or more selected from the group consisting of glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, and pharmaceutically acceptable salts thereof.

In one embodiment, the method comprises administering to the subject an effective amount of a composition comprising one or more selected from the group consisting of glipizide, meropenem, oxcarbazepine, doripenem, phenylbutazone, enoxacin, cilostazol, triamcinolone, didanosine, prazosin HCl, indomethacin, diethylstibestrol, emtricitabine, and pharmaceutically acceptable salts thereof.

In one embodiment, the method comprises administering to the subject an effective amount of a composition comprising one or more selected from the group consisting of phenylbutazone, meropenem, diethylstilbestrol, prazosin HCl, enoxacin, glipizide, doripenem hydrate, and pharmaceutically acceptable salts thereof.

In one embodiment, the method comprises administering to the subject an effective amount of a composition comprising one or more selected from the group consisting of phenylbutazone, prazosin HCl, enoxacin, and pharmaceutically acceptable salts thereof.

In certain embodiments, the subject has cancer, is diagnosed with cancer, or is suspected of having cancer. In certain embodiments, the subject has cancer is undergoing radiation therapy, or is planning to undergo radiation therapy. In certain embodiments, the cancer is head and neck cancer.

In one embodiment, the method comprises administering the composition to the subject before exposure to radiation therapy. Examples of administering the composition to the subject before exposure to radiation therapy include, but are not limited to, administering the composition 24 hours, 12 hours, 11 hours, 10 hours, 9 hours, 8 hours, 7 hours, 6 hours, 5 hours, 4 hours, 3 hours, 2.5 hours, 2 hours, 1.5 hours, 1 hour, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, 1 minute, and 30 seconds before exposure to radiation therapy. In one embodiment, the method comprises administering the composition to the subject more than one time before exposure to radiation therapy.

In some embodiments, the length of time the composition is administered to the subject prior to exposure to radiation therapy is dependent on the half-life of the one or more compounds in the composition. In some embodiments, the composition is administered less than about 3 half-lives, less than about 2.5 half-lives, less than about 2 half-lives, less than about 1.5 half-lives, less than about 1 half-life, less than about 0.9 half-lives, less than about 0.8 half-lives, less than about 0.7 half-lives, less than about 0.6 half-lives, less than about 0.5 half-lives, less than about 0.4 half-lives, less than about 0.3 half-lives, less than about 0.2 half-lives, less than about 0.1 half-lives, less than about 0.075 half-lives, less than about 0.05 half-lives, or less than about 0.025 half-lives before exposure to radiation therapy.

In one embodiment, the method comprises administering the composition to the subject during exposure to radiation therapy. In one embodiment, the method comprises administering the composition to the subject one or more times during exposure to radiation therapy. In one embodiment the method comprises administering the composition to the subject continuously during exposure to radiation therapy.

In one embodiment, the method comprises administering the composition to the subject after exposure to radiation therapy. Examples of administering the composition to the subject after exposure to radiation therapy include, but are not limited to, administering the composition 24 hours, 12 hours, 11 hours, 10 hours, 9 hours, 8 hours, 7 hours, 6 hours, 5 hours, 4 hours, 3 hours, 2.5 hours, 2 hours, 1.5 hours, 1 hour, 50 minutes, 45 minutes, 40 minutes, 35 minutes, 30 minutes, 25 minutes, 20 minutes 15 minutes, 10 minutes, 5 minutes, 4 minutes, 3 minutes, 2 minutes, 1 minute, and 30 seconds before exposure to radiation therapy. In one embodiment, the method comprises administering the composition to the subject more than one time after exposure to radiation therapy.

In one embodiment, the method comprises administering the composition to the subject before and during exposure to radiation therapy. In one embodiment, the method comprises administering the composition one or more times before exposure to radiation therapy and one or more times during radiation therapy. In one embodiment, the method comprises administering the composition once before exposure to radiation therapy and a second time during radiation therapy. In one embodiment, the method comprises administering the composition to the subject continuously starting before exposure to radiation therapy and continuing during part or all the radiation therapy.

In one embodiment, the method comprises administering the composition to the subject before and after exposure to radiation therapy. In one embodiment, the method comprises administering the composition to the subject one or more times before exposure to radiation therapy and administering the composition to the subject one or more times after exposure to radiation therapy.

In one embodiment, the method comprises administering the composition to the subject during and after exposure to radiation therapy. In one embodiment, the method comprises administering the composition to the subject one or more times before exposure to radiation therapy and administering the composition to the subject one or more times after exposure to radiation therapy. In one embodiment, the method comprises administering the composition to the subject continuously beginning during exposure to radiation therapy and ending after exposure to radiation therapy.

In one embodiment, the method comprises administering the composition to the subject before, during, and after exposure to radiation therapy. In one embodiment, the method comprises administering the composition to the subject one or more times before exposure to radiation therapy, administering the composition to the subject one or more times during exposure to radiation therapy, and administering the composition to the subject one or more times after exposure to radiation therapy. In one embodiment, the method comprises administering the composition to the subject continuously beginning before exposure to radiation therapy and ending after radiation therapy. In one embodiment, the method comprises administering the composition to the subject continuously beginning before exposure to radiation therapy and ending during radiation therapy and administering the composition to the subject continuously beginning during exposure to radiation therapy and ending after radiation therapy.

In some embodiments, the method comprises administering the composition to the subject before exposure to radiation therapy and eliminating the composition from the subject after exposure to radiation therapy. Examples of eliminating the composition from the subject after exposure to radiation therapy include, but are not limited to, eliminating the composition 24 hours, 12 hours, 11 hours, 10 hours, 9 hours, 8 hours, 7 hours, 6 hours, 5 hours, 4 hours, 3 hours, 2 hours, 1 hour, 45 minutes, 30 minutes, 15 minutes, 10 minutes, 5 minutes, 4 minutes, 3 minutes, 2 minutes, 1 minute, and 30 seconds after exposure to radiation therapy. Examples of eliminating the composition from the subject include, but are not limited to, dilution, chelation, filtration, metabolism, and excretion. In one embodiment, the method comprises administering the composition to the subject before exposure to radiation therapy and administering a second composition to the subject after exposure to radiation therapy which aids in, accelerate, or induces elimination of the composition administered before exposure to radiation therapy.

In some embodiments, the method comprises administering the composition to the subject during exposure to radiation therapy and eliminating the composition from the subject after exposure to radiation therapy. In one embodiment, the method comprises administering the composition to the subject during exposure to radiation therapy and administering a second composition to the subject after exposure to radiation therapy which aids in, accelerate, or induces elimination of the composition administered before exposure to radiation therapy.

The compositions of the disclosure may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation, or catheterization. Any administration may be a single application of a composition of invention or multiple applications. Administrations may be to single site or to more than one site in the individual to be treated. Multiple administrations may occur essentially at the same time or separated in time.

In some embodiments, the compositions may be administered in combination with one or more additional compositions. In certain embodiments, the composition of the present invention may be combined with one or more additional composition of the present invention.

In some embodiments, the method comprises administering an effective amount of a composition comprising two or more compounds selected from the group consisting of: glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, and pharmaceutically acceptable salts thereof. In some embodiments, the method comprises administering an effective amount of a composition comprising three or more compounds selected from the group consisting of: glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, and pharmaceutically acceptable salts thereof.

In some embodiments, the method comprises administering an effective amount of a composition comprising two or more compounds selected from the group consisting of glipizide, meropenem, oxcarbazepine, doripenem, phenylbutazone, enoxacin, cilostazol, triamcinolone, didanosine, prazosin HCl, indomethacin, diethylstibestrol, emtricitabine, and pharmaceutically acceptable salts thereof. In some embodiments, the method comprises administering an effective amount of a composition comprising three or more compounds selected from the group consisting of glipizide, meropenem, oxcarbazepine, doripenem, phenylbutazone, enoxacin, cilostazol, triamcinolone, didanosine, prazosin HCl, indomethacin, diethylstibestrol, emtricitabine, and pharmaceutically acceptable salts thereof.

In some embodiments, the method comprises administering an effective amount of a composition comprising two or more compounds selected from the group consisting of phenylbutazone, meropenem, diethylstilbestrol, prazosin HCl, enoxacin, glipizide, doripenem hydrate, and pharmaceutically acceptable salts thereof. In some embodiments, the method comprises administering an effective amount of a composition comprising three or more compounds selected from the group consisting of phenylbutazone, meropenem, diethylstilbestrol, prazosin HCl, enoxacin, glipizide, doripenem hydrate, and pharmaceutically acceptable salts thereof.

In some embodiments, the method comprises administering an effective amount of a composition comprising two or more compounds selected from the group consisting of meropenem, diethylstibestrol, glipizide, doripenem hydrate, and pharmaceutically acceptable salts thereof. In some embodiments, the method comprises administering an effective amount of a composition comprising three or more compounds selected from the group consisting of meropenem, diethylstibestrol, glipizide, doripenem hydrate, and pharmaceutically acceptable salts thereof. In some embodiments, the method comprises administering an effective amount of a composition comprising meropenem, diethylstibestrol, glipizide, and doripenem hydrate, or pharmaceutically acceptable salts thereof.

In certain embodiments, the composition of the invention may be combined with additional compounds that are useful for managing radiation treatment. Such compounds include, but are not limited to anesthetics, dyes, targeting agents, chelating agents, antibodies, or other compounds known to reduce the symptoms of radiation treatment.

Methods of the present invention are applicable to a variety of radiation therapies, including ionizing and non-ionizing radiation. Exemplary types of ionizing radiation for which the methods of the present invention may provide protection against include, but are not limited to, alpha, beta, gamma, positron, and X-ray radiation.

As is well known in the art, the effective dose of ionizing radiation varies with the type of tumor and stage of cancer that needs to be treated. The effective dose can also vary based on other treatment modalities being administered to the patient, for example chemotherapeutic treatments and surgical treatments, and whether the radiation is administered pre- or post-surgery.

In some embodiments, methods of the present invention are effective for protecting against radiation damage from therapeutic doses that are less than about 100 Gy, less than about 90 Gy, less than about 80 Gy, less than about 70 Gy, less than about 60 Gy, less than about 50 Gy, less than about 40 Gy, less than about 30 Gy, less than about 20 Gy, less than about 19 Gy, less than about 18 Gy, less than about 17 Gy, less than about 16 Gy, or less than about 15 Gy.

The therapeutic dose of radiation can be delivered in fractions. Fractionation refers to spreading out the total dose of radiation over time, for example, over days, weeks or months. The dose delivered in each fraction can be about 1-15 Gy per day. The treatment plan can include a fraction treatment one or more times per day, every other day, weekly, etc. depending on the treatment needs of each patient. For example, a hypofractionation schedule comprises dividing the total dose into several relatively large doses, and administering the doses at least one day apart.

The radiation treatment plan can include visualizing or measuring the tumor volume that needs to be irradiated, the optimal or effective dose of radiation administered to the tumor, and the maximum dose to prevent damage to nearby healthy tissue or organs at risk. Algorithms can used in treatment planning, and include dose calculation algorithms based on the particular radiotherapy technique parameters employed, e.g., gantry angle, MLC leaf positions, etc., and search algorithms which use various techniques to adjust system parameters between dose calculations to optimize the effectiveness of the treatment. Exemplary dose calculation algorithms include various Monte Carlo (“MC”) techniques and pencil beam convolution (“PBC”). Exemplary search algorithms include various simulated annealing (“SA”) techniques, algebraic inverse treatment planning (“AITP”), and simultaneous iterative inverse treatment planning (“SIITP”). Such techniques, and others, are well known in the art, and are included within the scope of this disclosure.

Radiation treatment planning algorithms may be implemented as part of an integrated treatment planning software package which provides additional features and capabilities. For example, a dose calculation algorithm and search algorithm may be used to optimize a set of fluence maps at each gantry angle, with a separate leaf sequencer used to calculate the leaf movements needed to deliver them. Alternatively, a dose calculation algorithm and search algorithm may be used to directly optimize leaf movements and other machine parameters.

Radiation therapy techniques that can be employed in conjunction with the methods of the invention include, but are not limited to, external-beam radiotherapy (“EBRT”) and Intensity Modulated Radiotherapy (“IMRT”), which can be administered by a radiotherapy system, such as a linear accelerator, equipped with a multileaf collimator (“MLC”). The use of multileaf collimators and IMRT allows the patient to be treated from multiple angles while varying the shape and dose of the radiation beam, thereby avoiding excess irradiation of nearby healthy tissue. Other exemplary radiation therapy techniques include stereotactic body radiotherapy (SBRT), volumetric modulated arc therapy, three-dimensional conformal radiotherapy (“3D conformal” or “3DCRT”), image-guided radiotherapy (IGRT). The radiation therapy techniques can also include Adaptive radiotherapy (ART), a form of IGRT that can revise the treatment during the course of radiotherapy in order to optimize the dose distribution depending on patient anatomy changes, and organ and tumor shape. Another radiation therapy technique is brachytherapy. In brachytherapy, a radioactive source is implanted within the body of the subject, such that the radioactive source is near the tumor. As used herein, the term radiotherapy should be broadly construed and is intended to include various techniques used to irradiate a patient, including use of photons (such as high energy x-rays and gamma rays), particles (such as electron and proton beams), and radiosurgical techniques.

### Pharmaceutical Compositions and Formulations

The invention also encompasses the use of pharmaceutical compositions to practice the methods of the invention. Such a pharmaceutical composition may consist of at least one composition of the invention or a salt thereof in a form suitable for administration to a subject, or the pharmaceutical composition may comprise at least one composition of the invention or a salt thereof, and one or more pharmaceutically acceptable carriers, one or more additional ingredients, or some combination of these. The compound or conjugate may be present in the pharmaceutical composition in the form of a physiologically acceptable salt, such as in combination with a physiologically acceptable cation or anion, as is well known in the art.

Pharmaceutical compositions that are useful in the methods of the invention may be suitably developed for oral, rectal, vaginal, parenteral, topical, pulmonary, intranasal, buccal, sublingual, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

In some embodiments, a pharmaceutical composition of the present invention is formulated for local delivery via injection. In some embodiments, the pharmaceutical composition is designed for sustained release at the delivery site. In some embodiments, the composition comprises a microsphere, microparticle, nanosphere, nanoparticle, biodegradable gel, hydrogel, matrix, or polymer which releases one or more compounds over time to the localized area. Examples of injectable liquid polymer compositions suitable for local delivery in methods of the invention can be found in the art (e.g., U.S. patent application Ser. No. 17/278,930).

Although the invention herein is principally directed to the ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions is contemplated include, but are not limited to, humans and other primates, mammals including commercially relevant mammals such as cattle, pigs, horses, sheep, cats, rodents, and dogs.

In one embodiment, the compositions utilized in the invention are formulated using one or more pharmaceutically acceptable excipients or carriers. In one embodiment, the pharmaceutical compositions comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In one embodiment isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition utilized in the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an anti-oxidant and a chelating agent that inhibits the degradation of the compound. Exemplary antioxidants for some compounds are BHT, BHA, alpha-tocopherol and ascorbic acid in the range of about 0.01% to 0.3%. In one embodiment, the BHT is in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In one embodiment, chelating agents may be in the range of 0.02% to 0.10% by weight by total weight of the composition. The chelating agent is useful for chelating metal ions in the composition that may be detrimental to the shelf life of the formulation. While BHT and disodium edetate are the exemplary antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition for use in the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

In some embodiments, the liquid solution is a viscous gel. In some embodiments, the gel composition comprises one or more gelling agents. Examples of gelling agents include, but are not limited to, poloxamers, chitosan, methylcellulose, ethylcellulose, propylcellulose, carboxymethylcellulose, hydroxyethylcellulose, hydroxymethylcellulose, hydroxypropylcellulose, pectin, xylulose, Carbopol, guar gum, gellan gum, xanthan gum, gum acacia, pullulan, tragacanth, starch, carbomer, alginates, gelatin, polyvinylacohols, bentonite, carrageenan, hyaluronic acid, polyethylene oxide, polypropylene oxide, and polycarbophil. In some embodiments, the gel composition comprises one or more compounds which accelerate gelation. Examples of acceptable compounds include, but are not limited to, calcium chloride, calcium bromide, calcium iodide, and calcium lactate.

In some embodiments, the composition is an injectable liquid polymer composition. Injectable liquid polymer compositions may comprise one or more biodegradable polymers. Examples of biodegradable polymers include, but are not limited to, polyglycolides, polylactides, polycaprolactones, polyanhydrides, polyorthoesters, polydioxanones, polyacetals, polyesteramides, polyamides, polyurethanes, polycarbonates, polyphosphazenes, polyketals, polyhydroxybutyrates, polyhydorxyvalerates, polyhyaluronic acid, polyalkylene oxalates, and polyalkylene oxides. In some embodiments, the injectable liquid polymer composition comprises one or more biocompatible solvents and/or co-solvents. Examples of biocompatible solvents include, but are not limited to, α-tocopherol, acetone, acetyl tributylcitrate, acetyl triethyl citrate, benzyl alcohol, butanol, butyrolactone, caprolactone, castor oil, n-cyclohexyl-2-pyrrolidone, diethylene glycol monomethyl ether, dimethyl acetamide, dimethyl formamide (DMF), dimethyl sulfoxide (DMSO), ethyl acetate, ethyl lactate, N-ethyl-2-pyrrolidone, glycerol, glycofulol, hydrogenated castor oil, isobutanol, isopropanol, N-hydroxyethyl-2-pyrrolidone, isopropylidene glycerol, lactic acid, lauric acid, laurate esters, smethoxypolyethylene glycol, methoxypropylene glycol, methyl acetate, methyl ethyl ketone, methyl lactate, N-methyl-2-pyrrolidone (NMP), oleic acid, oleate esters, polyethylated castor oil, polyethylated hydrogenated castor oil, low-molecular weight polyethylene glycol (PEG), low-molecular weight polysorbates (e.g., 20, 40, 60, or 80), propanol, propylene glycol, 2-pyrrolidone, sorbitan monolaurate, sorbitan monooleate, sorbitan monostearate, stearic acid, stearoyl esters, triacetin, tributyl citrate, and triethyl citrate. Examples of injectable liquid polymer compositions suitable for use in the invention can be found in the art (e.g., U.S. patent application Ser. No. 17/278,930).

Powdered and granular formulations of a pharmaceutical preparation of the composition utilized in the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition for use in the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, such a mammal, including a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound for use in the invention is from about 1 and 5,000 mg/kg of body weight/per day. One of ordinary skill in the art would be able to study the relevant factors and make the determination regarding the effective amount of the therapeutic compound without undue experimentation.

The invention may be practiced as frequently as several times daily, or it may be practiced less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg/kg day dose may be initiated on Monday with a first subsequent 5 mg/kg per day dose administered on Wednesday, a second subsequent 5 mg/kg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In certain embodiments, the composition of the present invention provides for a controlled release of a therapeutic agent. In certain instances, controlled- or sustained-release formulations of a pharmaceutical composition of the invention may be made using conventional technology, using for example proteins equipped with pH sensitive domains or protease-cleavable fragments. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, lozenges, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased subject compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In certain embodiments, the controlled-release formulation of the composition described herein allows for release of a therapeutic agent precisely when the agent is most needed. In another embodiment, the controlled-release formulation of the composition described herein allows for release of a therapeutic agent precisely in conditions in which the therapeutic agent is most active. In order to maintain this constant level of drug in the body, the drug must be released from the dosage form at a rate that will replace the amount of drug being metabolized and excreted from the body.

In certain embodiments, the composition provides for an environment-dependent release, when and where the therapeutic agent is triggered for release. For example, in certain embodiments the composition invention releases at least one therapeutic agent when and where the at least one therapeutic agent is needed. The triggering of release may be accomplished by a variety of factors within the microenvironment of the treatment or prevention site, including, but not limited to, temperature, pH, the presence or activity of a specific molecule or biomolecule, and the like.

Controlled-release of an active ingredient can be stimulated by various inducers, for example pH, temperature, enzymes, water or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compositions are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

In one embodiment, the invention is practiced in dosages that range from one to five times per day or more. In another embodiment, the invention is practiced in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Routes of administration of include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In some embodiments, the route of administration is direct injection to the tissue to be protected from radiation damage. In some embodiments, the route of administration is direct injection to the salivary glands.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### EXPERIMENTAL EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Without further description, it is believed that one of ordinary skill in the art can, using the preceding description and the following illustrative examples, make and utilize the present invention and practice the claimed methods. The following working examples therefore, point out specific embodiments of the present invention, and are not to be construed as limiting in any way the remainder of the disclosure.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Example 1: High-Throughput Methods to Assess Drug Radioprotection

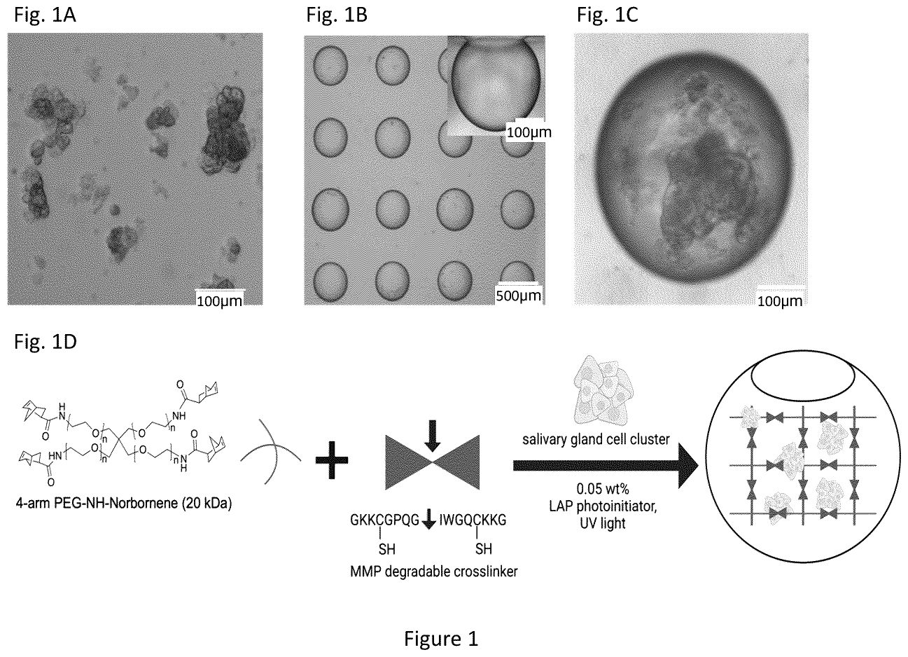

A salivary gland tissue chip was leveraged for high-content drug screening to identify novel radioprotective compounds (Song Y et al., Commun Biol., 2021, 4). The chip platform consists of an array of near-spherical microbubble (MB) cavities formed in poly(dimethylsiloxane) (PDMS). Each chip, containing ~50 MBs, affixed within wells of a 96-well plate (FIG. 1C). Primary salivary gland cell clusters (FIG. 1A), suspended in a poly(ethylene glycol) (PEG) hydrogel precursor and MMP-degradable peptide crosslinker solution together with the photoinitiator LAP, were applied to the chip allowing the clusters to deposit into the MBs (FIG. 1D). In situ polymerization of the hydrogel was achieved using long-wave, low intensity UV light. Over time, the cell clusters aggregate and proliferate to form SGm (FIG. 1C).

In prior studies, immunohistochemical (IHC) staining was used to quantify the number γH2AX and 53BP1 puncta within nuclei, which are sensitive markers for double-strand breaks (Djuzenova, C. S. et al. BMC Cancer 15, 856 (2015)) and, thus, direct measures of radiation damage/protection. IHC staining is, however, a laborious time and resource-consuming process requiring retrieval of the SGm from the MB array chip, tissue sectioning, staining, and imaging. While IHC enables sensitive analyses of radiation damage, it completely abrogates the goal of in situ high-content screening for which the tissue chip was developed. Several assays commonly used to assess radiation-induced cellular damage were tested in the tissue chip format at various time points post-radiation (Table 1). Based on signal-to-noise ratio and reproducibility, glutathione and senescence assays were selected for further development (Meeks, L et al., Physiol Genomics 53, 85-98 (2021); Peng, X. et al., Cell Death Dis 11, 854 (2020).

TABLE 1
Assays tested for detecting radioprotection in the salivary gland tissue chip.

| Assay | Category | Target | Time Points |
|---|---|---|---|
| Image-iT Lipid | Free radical | Lipid | 1-5 days |
| peroxidation | damage | peroxidation | |
| CellEvent caspase 3/7 | Apoptosis | Caspase 3/7 | 2-7 days |
| CellROX | Free radicals | ROS | 1-3 days |
| Calcein AM | Cell viability | Esterase activity | 0.5-2 hours, |
| | | | 2-4 days |
| PrestoBlue | Metabolism | NAD⁺/NADH | 0.5-6 hours, |
| | | | 2-4 days |
| CellTiterFluor | Metabolism | Aminopeptidase | 0.5-6 hours, |
| | | | 2-4 days |
| Carbonic anhydrase | Secretion | Carbonic | 2 hours, |
| assay kit | | anhydrase | 2-7 days |
| Calcium signaling | Secretion | Calcium | 24 hours |
| | | signaling | |
| | | response to | |
| | | agonists | |
| PAS-AB stain | Secretion | Mucins | 2-7 days |
| H2DCFDA (2′7′- | Free radicals | ROS | 1-8 hours, |
| dichlorohydrofluorescein | | | 2-4 days |
| diacetate) | | | |
| Cellular glutathione | Antioxidant | Reduced | 0.5-6 hours, |
| detection kit* | | glutathione | 2-4 days |
| Cellular Senescence | Cellular | SA-ß- | 5 days, |
| Detection Kit - | senescence | galactosidase | 7 days |
| SPIDER-ßGal* | | | |

*Capable of detecting reliable changes between 0 Gy and 15 Gy at the bolded timepoints.

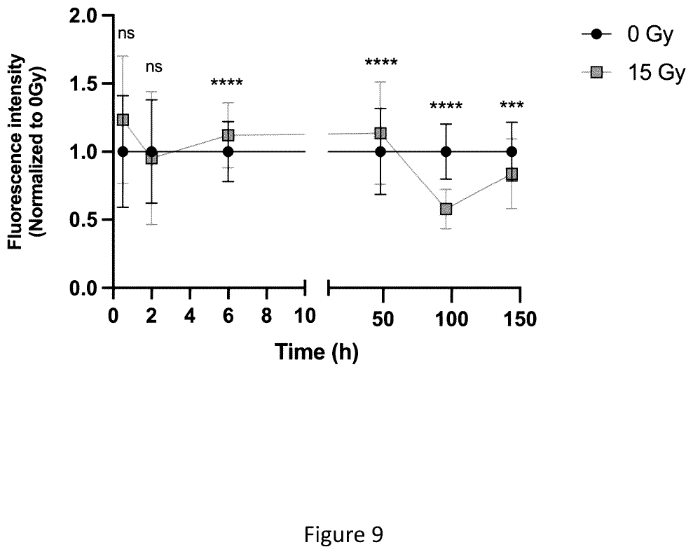

The glutathione assay was used to measure glutathione levels at various time points post-radiation to determine the optimal time point for detecting differences between 0 Gy and 15 Gy. Based on the data, the greatest signal separation was measured at 4 days post-irradiation (FIG. 9). This timepoint is similar to previous reports on decreases in glutathione post-radiation and was used for all experiments moving forward (Meeks L et al., Physiological Genomics, 2021, 53: 85-98).

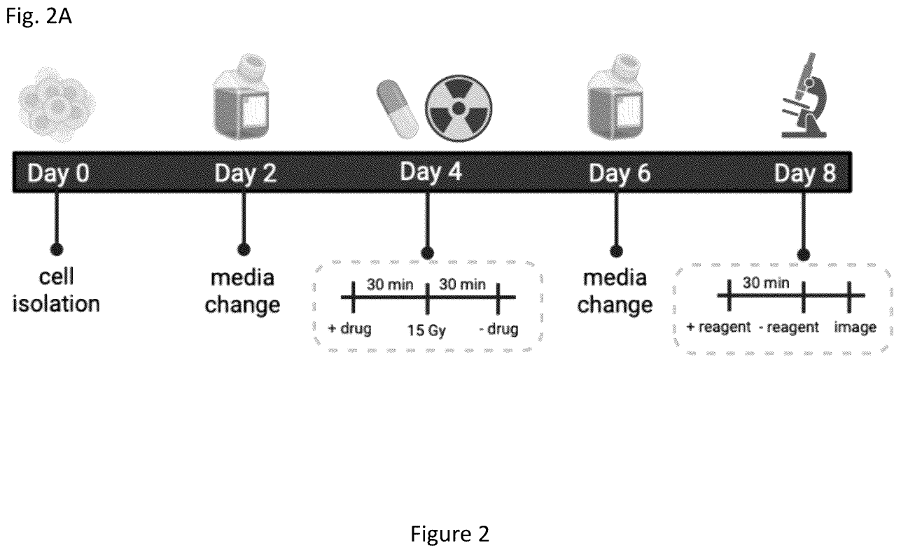

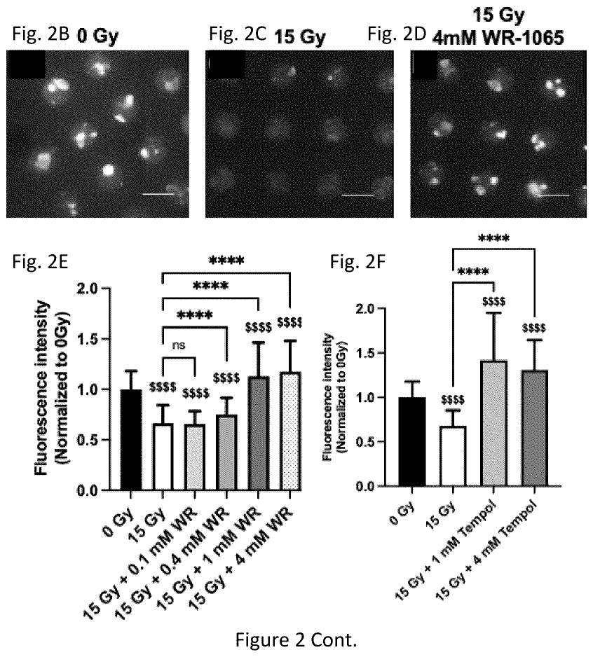

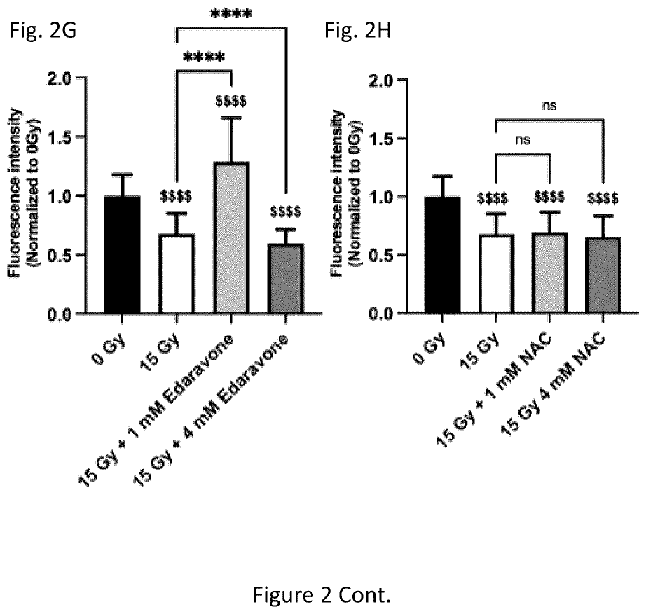

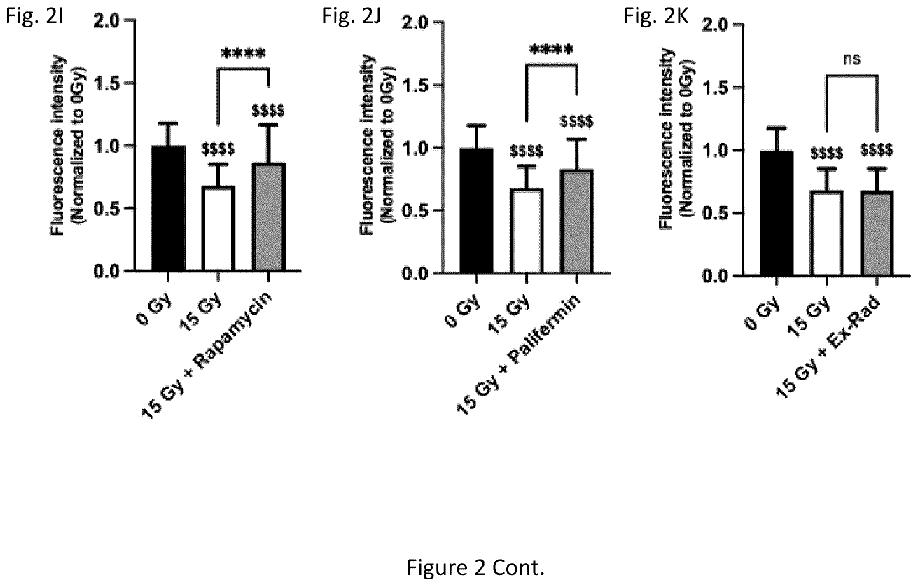

The dose response to WR-1065 was examined to determine effective concentrations of WR-1065 to prevent the reduction of glutathione levels post-radiation. A SGm tissue chip was cultured for 4 days to allow spheres to form. The chip was then treated with WR-1065 30 minutes before and during radiation, followed by drug wash out with media 30 minutes post-radiation (FIG. 2A). This dosing scheme is consistent with the use of Amifostine clinically and with previous work. The glutathione assay was performed at 4 days post-radiation (FIG. 2A). Example images show high levels of glutathione at 0 Gy (FIG. 2B) that is decreased by 15 Gy radiation (FIG. 2C) and maintained with 4 mM WR1065 (FIG. 2D). Quantification of glutathione levels demonstrates that doses of 0.1 mM and 0.4 mM WR-1065 were ineffective at preventing radiation damage-induced reduction of glutathione levels, while both 1 mM and 4 mM WR-1065 provided significant protection (FIG. 2E). The 4 mM dose corresponds with previous work on DNA damage markers γH2AX and 53BP1 and values from literature and establishes the range of effective concentrations of WR-1065 for in vitro treatment (Song Y et al., Commun Biol., 2021, 4; Hofer M et al., J Medicinal Chem, 2016, 59: 3003-3017). Moreover, it is clinically relevant as 15-30 minutes before radiation, patients are administered Amifostine intravenously at 200 mg/m (Escobar A et al., IntechOpen, 2019, pp. 15-37; Eisbruch A, J Clin Oncol, 2011, 29: 119-121; Bardet, E. et al. Semin Oncol, 2002, 29, 57-60). Assuming an average adult body surface area of 17,000 cm² and a blood volume of 1.35 gallons (5.1 liters), Amifostine is administered at 300 μM (Mosteller, R. D. N Engl J Med 317, 1098 (1987)).

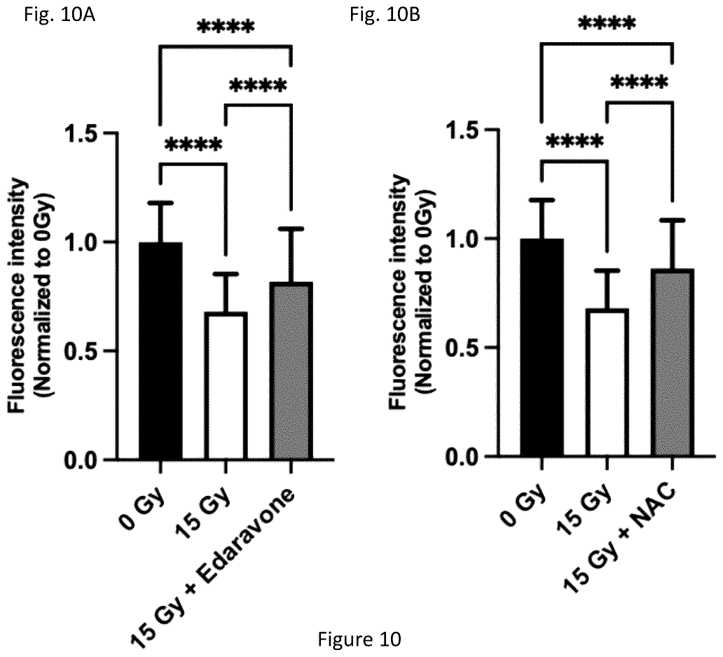

Since WR-1065 is an antioxidant and mediates radioprotective effects through free radical scavenging and induction of superoxide dismutase expression, other antioxidants implicated as radioprotective were tested (Tempol, Edaravone, N-acetylcysteine) (Noaparast Z et al., Future Oncol, 2013, 9: 1145-1159). Tempol exhibited strong radioprotection at both at 1 and 4 mM (FIG. 2F), which is consistent with prior studies (Cotrim A P et al., Clin Cancer Res, 2007, 13: 4928-4933; Vitolo J M et al., Clin Cancer Res, 2004, 10: 1807-1812; Citrin D et al., The Oncologist, 2010, 15: 360-371). Edaravone showed protection at 1 mM but not at 4 mM (FIG. 2G). Edaravone maintained ~43% of glutathione levels at 0.1 mM (FIG. 10A); suggesting that the optimal concentration range for Edaravone might be lower than that of WR-1065, which is suggested by literature indicating radio-protective dose ranges are 100-1000 μM (Chen L et al., Cell Stress and Chaperones, 2015, 20: 289-295; Homma T et al., Experimental Cell Research, 2019, 384: 111592; Ishii J et al., Neuroscience Letters, 2007, 423: 225-230). For N-acetylcysteine (NAC), no improvement in glutathione levels was observed at 1 or 4 mM concentration compared to untreated SGm (FIG. 2H); however, glutathione levels were rescued by 56% when treated with 10 mM NAC (FIG. 10B), consistent with literature (Kim H J et al., Cancer Res Treat, 2020, 52: 1019-1030).

Drugs reported with non-antioxidant radioprotective mechanisms were also tested using the glutathione assay. Rapamycin, an inhibitor of mTOR, has been reported to restore salivary flow rate post-radiation in swine (Zhu Z et al., Oncotarget, 2016, 7: 20271-20281). Ex-Rad has been shown to reduce p53-dependent and independent apoptosis (Ghosh S P et al., Radiation Res, 2009, 171: 173-179). Palifermin (keratinocyte growth factor) has been reported to stimulate salivary gland stem/progenitor cell expansion post-radiation (Lombaert I M A et al., Stem Cells, 2008, 26: 2595-2601). Using drug concentrations based on literature, the data shows some protection against glutathione level changes resulting from radiation with 50 μM rapamycin (58%; FIG. 2I) and 100 ng/mL Palifermin (47%; FIG. 2J) with no effect observed for 50 μM Ex-Rad (FIG. 2K).

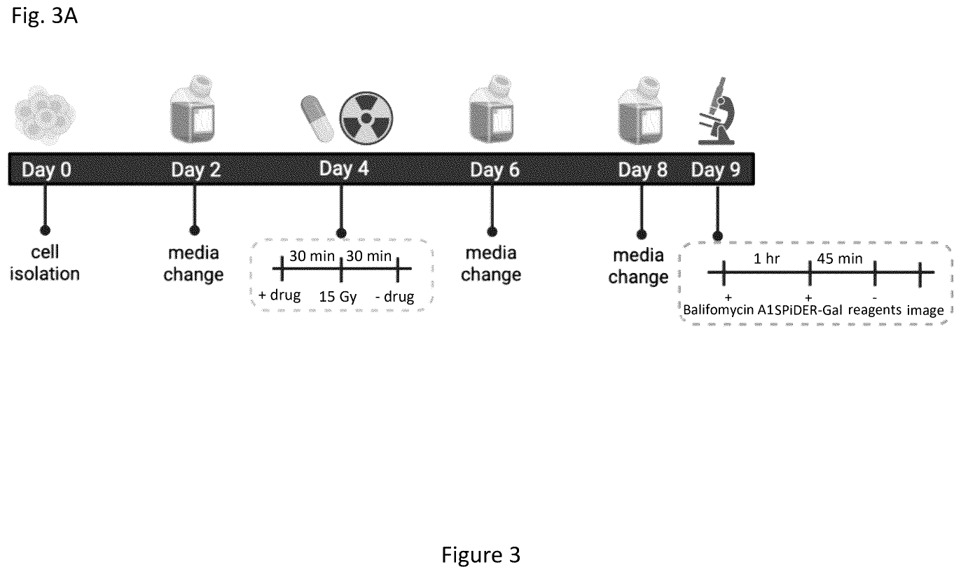

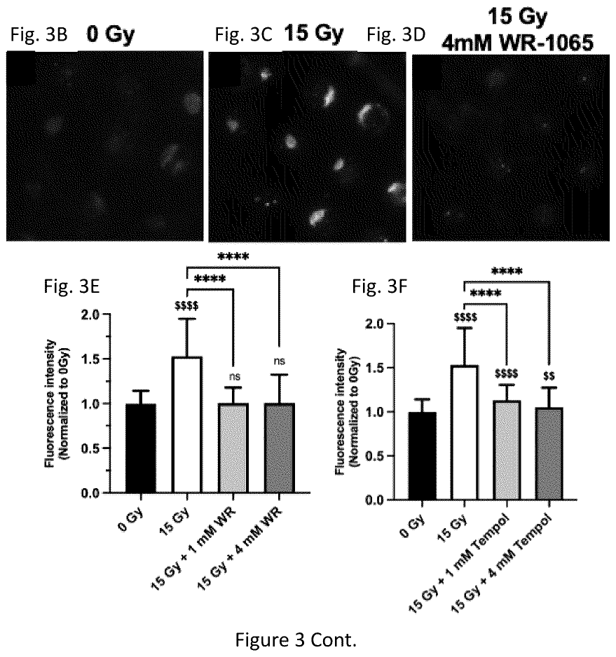

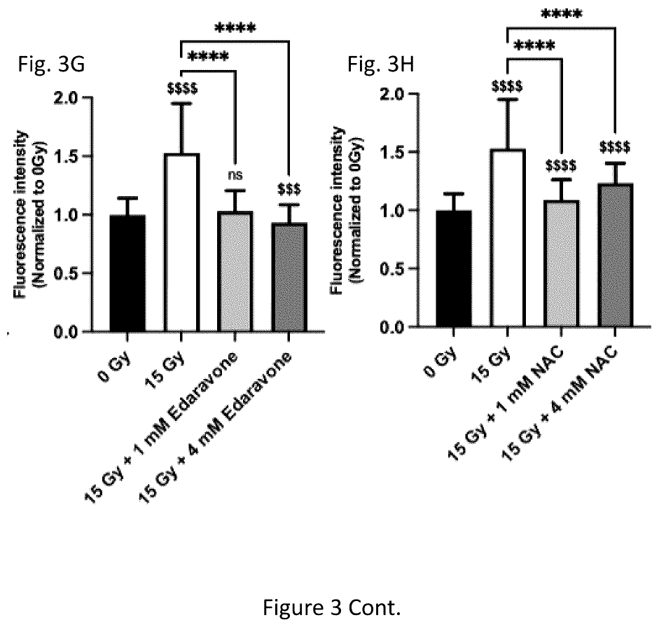

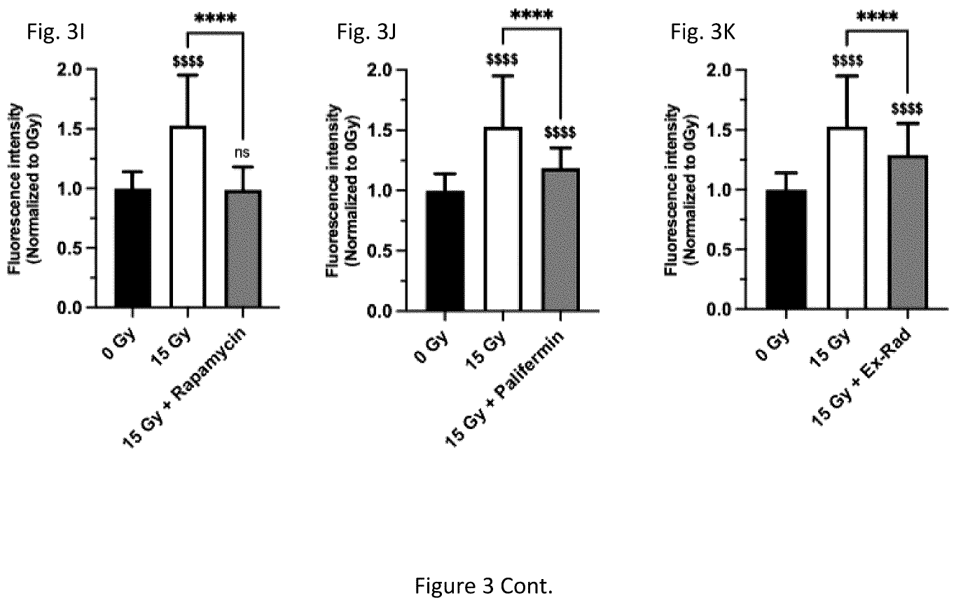

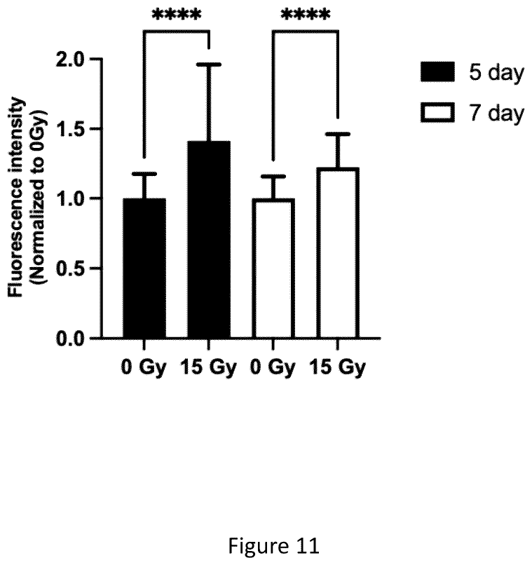

For senescence, a protocol similar to the glutathione assay was followed to determine the optimal time point post-radiation for detecting a change in senescence between 0 Gy and 15 Gy, as measured by senescence-associated β-galactosidase activity. A time point of 5 days post-radiation was optimal (FIG. 11), similar to a previous study. WR-1065 radioprotection was tested by adding drug to the chips 30 min before radiation followed by wash out 30 min post-radiation (FIG. 3A) (Peng X et al., Cell Death and Disease, 2020, 11: 854). An expected increase in senescence was detected for SGm exposed 15 Gy compared to 0 Gy (FIG. 3B-C) which was restored to levels equivalent to 0 Gy with the addition of WR-1065 (FIG. 3D). Quantification shows that both 1 and 4 mM WR-1065 resulted in complete radioprotection (FIG. 3E), consistent with the results from the glutathione assay.

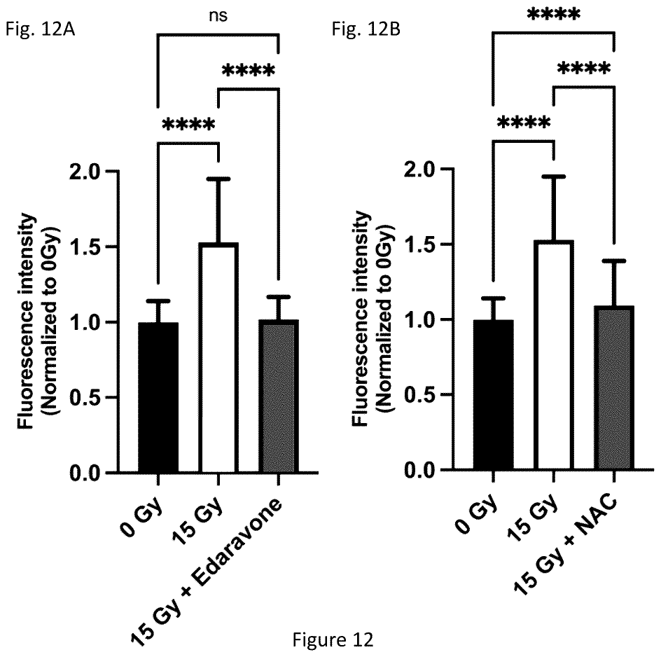

The senescence assay was then performed with the other radioprotective drugs. Tempol and Edaravone reduced senescence at both 1 mM and 4 mM by 75% and 91% for Tempol, 94% and 113% for Edaravone, at 1 and 4 mM, respectively versus untreated, irradiated controls (FIG. 3F-G). Edaravone also reduced senescence by 96% at 100 μM versus untreated, irradiated controls (FIG. 12A). For NAC, 83% and 57% reduction in senescence was observed with 1 mM and 4 mM (FIG. 3H) and 82% at 10 mM (FIG. 12B) versus untreated, irradiated controls. For drugs with non-antioxidant mechanisms, rapamycin showed complete protection (FIG. 3I), whereas Palifermin provided 64% protection (FIG. 3J) and Ex-Rad conferred only 45% radioprotection (FIG. 3K).

A summary of results from the glutathione and senescence assays shows similar trends for radioprotection (Table 7). The few differences may result from different mechanisms of action and/or assay targets. Logically, the glutathione assay may be more sensitive to antioxidant function, while the senescence assay may be more appropriate for drugs like rapamycin, which has anti-senescence properties (Lombaert I M A et al., Stem Cells, 2008, 26: 2595-2601; Iglesias-Bartolome R et al., Cell Stem Cell, 2012, 11: 401-414). These results highlight the trade-offs in developing screening assays and point to the benefit of screening with two assays. Although the assays developed are indirect measures of radiation-induced DNA damage, they nonetheless were validated to detect radiation induce cell-damage and drug radioprotection. Moreover, these assays can be used for in situ high-content drug screening with multiple replicates (40-50) per test and enhanced throughput compared to immunohistochemical staining for γH2AX.

TABLE 7
Results of known radioprotective drugs in glutathione and senescence assays.

| Drug | Concentration | Glutathione | Senescence |
|---|---|---|---|
| WR-1065 | 1 mM | + | + |
| | 4 mM | + | + |
| Tempol | 1 mM | + | + |
| | 4 mM | + | + |
| Edaravone | 100 μM | ~ | + |
| | 1 mM | + | + |
| | 4 mM | — | + |
| N-acetylcysteine | 1 mM | — | ~ |
| | 4 mM | — | ~ |
| | 10 mM | ~ | ~ |
| Rapamycin | 50 μM | ~ | + |
| Palifermin | 100 ng/ml | ~ | ~ |
| Ex-Rad | 50 μM | — | ~ |

+, radioprotection; ~ , partial radioprotection; — no radioprotection.

### Example 3: Selleck Chemicals Library Screening

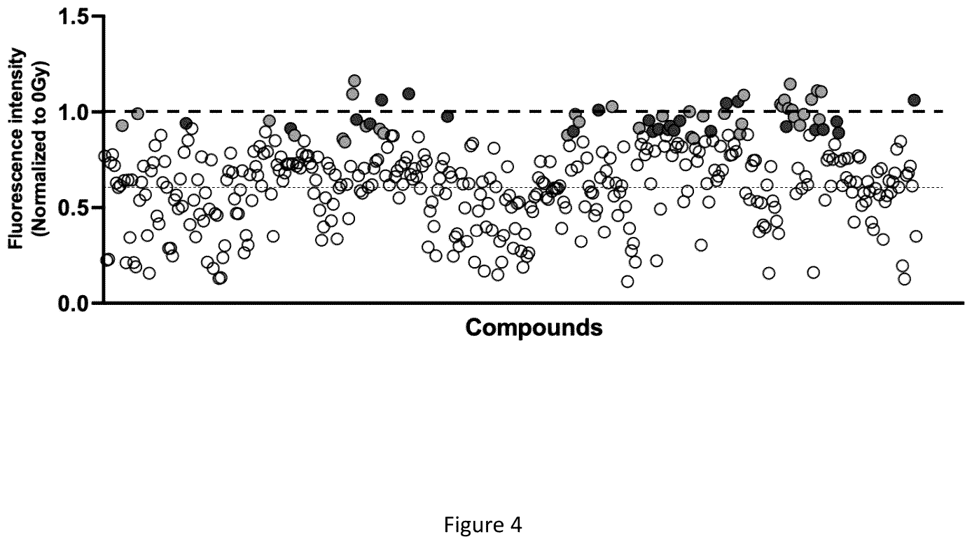

The glutathione and senescence assays were used to screen a library of FDA-approved drugs (Selleck Chemicals) at 100 μM. Drugs were first screened using the glutathione assay according to the timeline depicted in FIG. 2A. Any compound resulting in statistically similar glutathione levels compared to 0 Gy control was considered a hit (FIG. 4, grey circles). Hits with the glutathione assay were then tested with the senescence assay and considered a “double hit” if senescence levels were statistically similar to levels at 0 Gy (FIG. 4, black circles). A list of the 438 drugs screened and relevant statistics are shown in Table 7. Overall, 438 drugs from the library were tested with a hit rate of 5.7% for a total of 25 double hits (Table 2) listed in the Table 3. While this hit rate is higher than many other drug screening reports (0.1-0.3%), this may be due to the statistical rigor afforded by the tissue chip format (Blanchard, C et al., Antimicrob Agents Chemother 2016, 60, 862-872; Colquhoun, J. M et al., Antibiotics (Basel) 2019, 8, 48). Additionally, phenotypic screens generally have higher hit rates than target-based screens (>1%) (Moffat, J. G et al., Nature Reviews Drug Discovery 2017, 16, 531-543; Rottman, M et al., Science 2010, 329, 1175-1180).

TABLE 2
Total and percent positive compounds using the initial glutathione
assay and combined glutathione and senescence assays]]

| | |
|---|---|
| Drugs screened with glutathione assay | 438 |
| Hits with glutathione assay | 62 |
| Hit rate (%) | 14.2 |
| Double hits | 25 |
| Double hit rate (%) | 5.7 |

TABLE 3
Drug activity based on data from the BioAssay database.

| Drug | Total Assays | Active (%) |
|---|---|---|
| Glipizide (Glucotrol) | 3141 | 0.5 |
| Etidronate (Didronel)† | 1402 | 1.1 |
| Meropenem | 7031 | 1.3 |
| Oxcarbazepine | 2774 | 1.3 |
| Doripenem | 2434 | 1.4 |
| Phenylbutazone | 5565 | 1.5 |
| Enoxacin (Penetrex) | 2673 | 1.9 |
| Cilostazol | 3093 | 2.5 |
| Triamcinolone (Aristocort) | 2345 | 2.7 |
| Didanosine (Videx) | 280 | 5.7 |
| Eplerenone† | 610 | 5.9 |
| Prazosin HCl | 1264 | 6.3 |
| Indomethacin | 10138 | 6.6 |
| Diethylstilbestrol (Stilbestrol) | 5048 | 7.2 |
| Melatonin† | 4488 | 8.2 |
| Emtricitabine (Emtriva) | 516 | 9.3 |
| Albendazole (Albenza)† | 3086 | 9.3 |
| Rifampin* | 8071 | 11.0 |
| Nifedipine (Adalat)* | 6190 | 11.5 |
| Rifabutin (Mycobutin)* | 1123 | 12.0 |
| Calcitriol (Rocaltrol) * | 1786 | 15.2 |
| Lomustine (CeeNU)* | 5832 | 16.1 |
| Adefovir Dipivoxil (Preveon)* | 929 | 22.7 |
| Curcumin* | 3404 | 29.7 |
| Gemcitabine (Gemzar) | 2190 | 46.7 |

*, drugs were excluded from further analysis due to bioactivity in >10 % of assays.
†, Drugs were excluded based on poor bioavailability and batch variability (eplerenone).

### Example 4: Drug Down-Selection Process

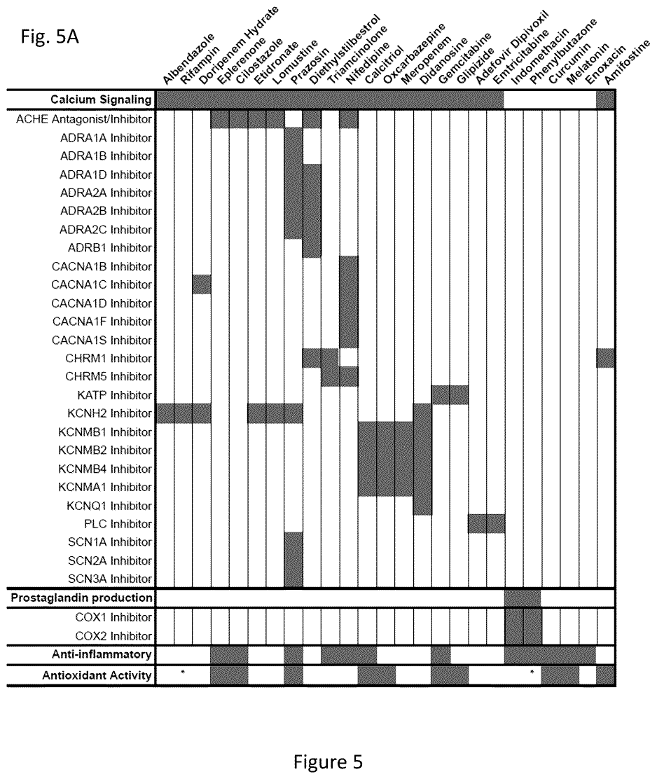

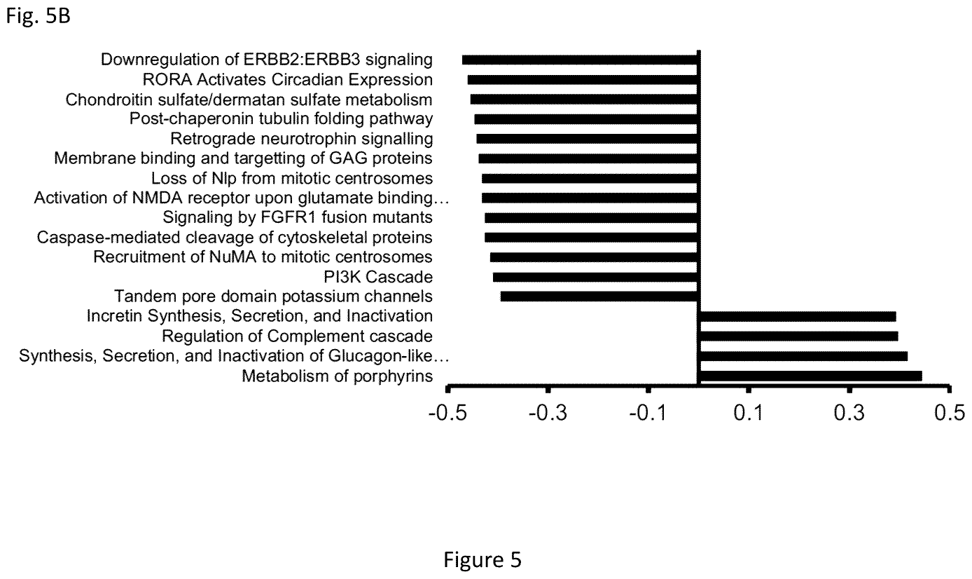

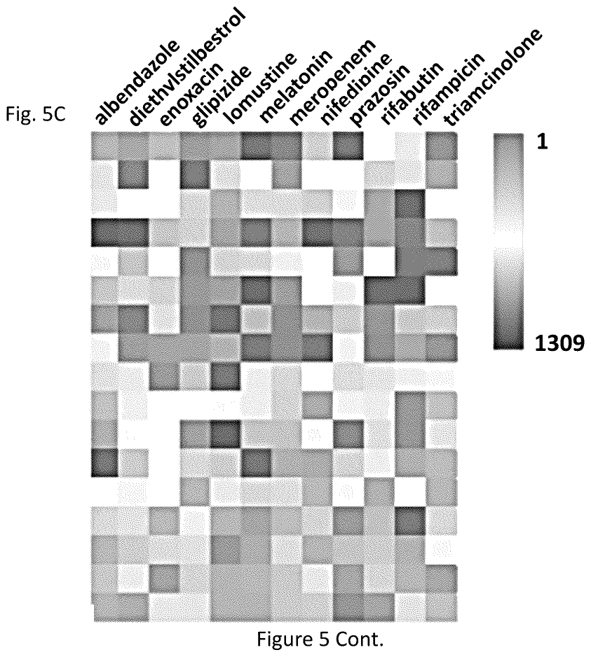

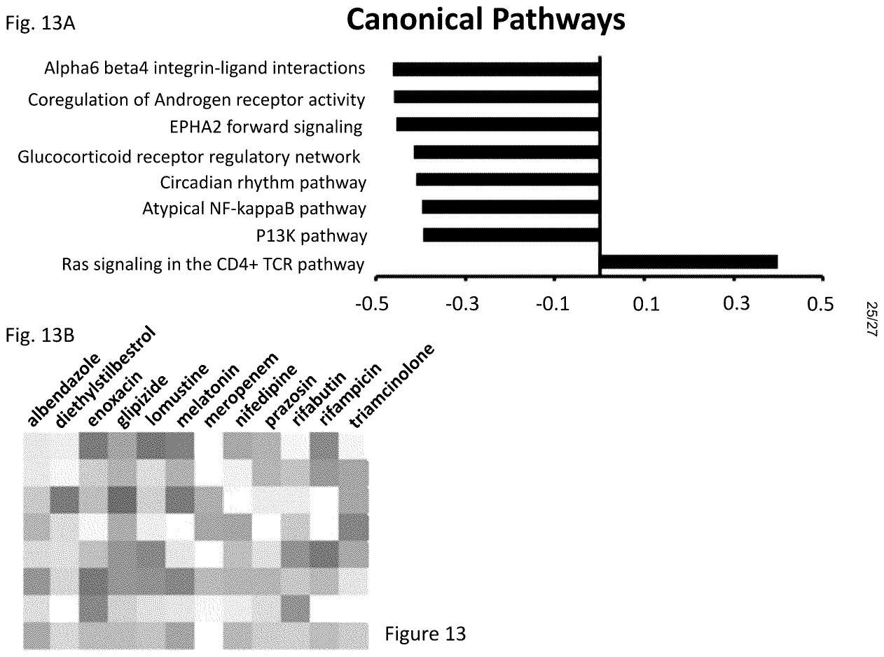

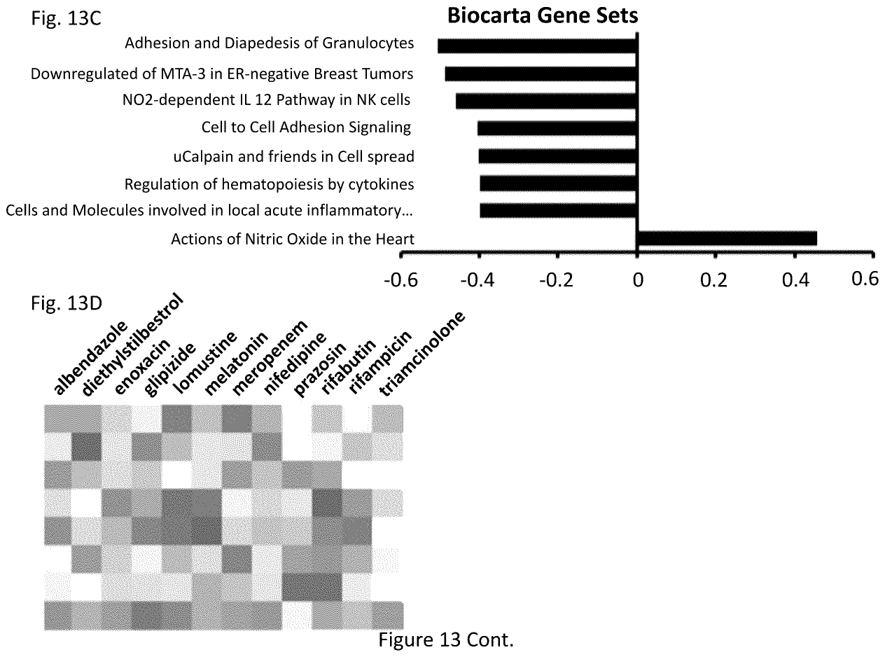

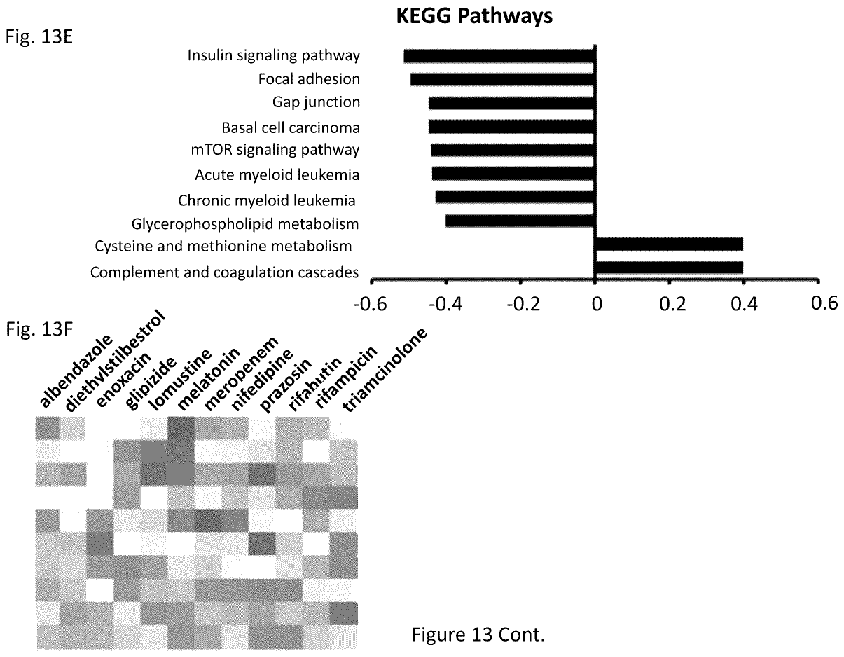

Of the 25 potential radioprotective compounds, 20 have known interactions with proteins involved in calcium signaling identified within the BioAssay database in PubChem (FIG. 5A). These compounds may impact secretory signaling in the salivary gland, which may be radioprotective. While degranulation may not be key to radioprotection, secretory stimulation may play a role in proliferation and survival of the secretory cells (Coppes, R. P. et al., Radiat Res 148, 240-247 (1997)). Similarly, using the Drug Set Enrichment Analysis (DSEA) tool to identify pathways, the Reactome analysis related to secretion appear to be upregulated by many of the identified drugs, supporting this potential mechanism (FIG. 5B-C, and FIG. 13). Interestingly, only 9 of the 25 compounds have known antioxidant properties, and 12 are anti-inflammatory. This is critical data indicating that alternate mechanisms of radioprotection may be achievable and represented in the identified hits. A reduction in pathway activity related to cell adhesion, cell-cell and cell-matrix interactions also represents a potential area of exploration. Multiple studies have identified changes in integrin expression, cell adhesion and matrix interactions upon radiation exposure which may be linked to cell response (Jasmer, K. J. et al., J Clin Med 9 (2020); Lafrenie, R. M. et al., Annals of the New York Academy of Sciences 842, 42-48 (1998); Rose, R. W. et al., Radiat Res 152, 14-28 (1999); Cordes, N. et al., Int J Radiat Biol 78, 347-357 (2002); Baluna, R. G. et al., Radiat Res 166, 819-831 (2006)). Manipulation of these mechanisms in the salivary gland may convey radioprotection.

A systematic approach was used to down selection the 25 double hits for in vivo testing. Since the drugs in the library are FDA-approved, considerable information on their pharmacology in mice and humans is readily available through resources such as PbuChem. Within PubChem, the BioAssay database was created by the National Institutes of Health (NIH) as an open repository containing results of small molecule screening data and small interfering RNAs (siRNAs) screening (Wang, Y et al., Nucleic Acids Research 2010, 38, D255-D266). BioAssay results were used to analyze drug promiscuity, the ability of a drug to bind multiple molecular targets with distinct pharmacological outcomes, often causing unwanted side effects. As promiscuity of drugs often results in undesirable side effects, drugs exhibiting bioactivity in a large number of assays were deprioritized (Gupta, M. N et al., Biochimie 2020, 175, 50-57). Data for each of the double hits was obtained from the database and promiscuity was calculated as the percent of assays reported as “active” (Table 3). Drugs with a high percent (>10%) were excluded from further testing. Additionally, etidronate, melatonin, and albendazole were excluded due to poor bioavailability, and eplerenone was excluded due to concerns with solubility (DeMuro, R et al., J Clin Pharmacol 2000, 40, 781-784; Dunn, C et al., Drugs Aging 1994, 5, 446-474; Ochoa, D et al., Front Pharmacol 2021, 12, 6644665).

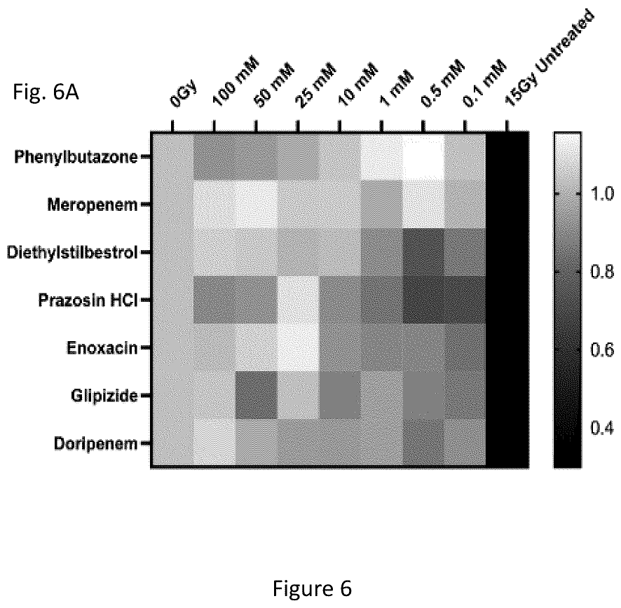

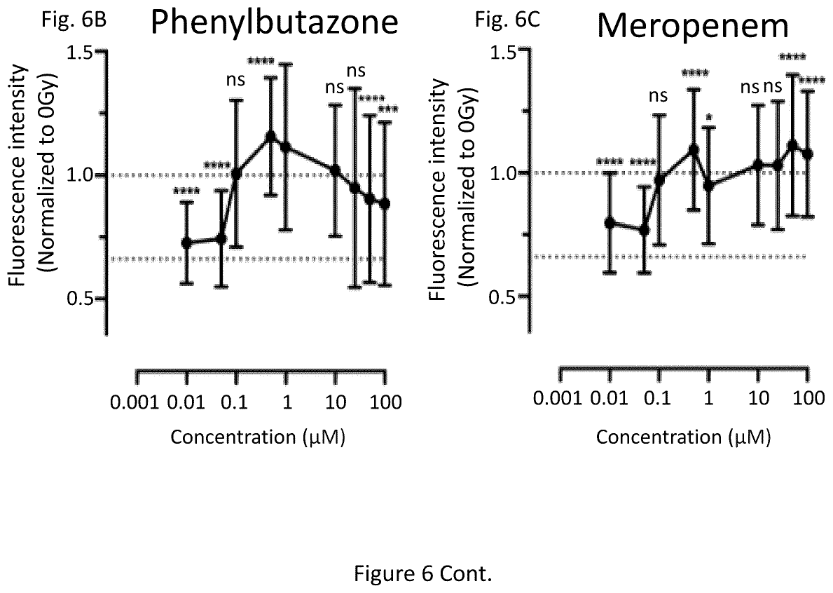

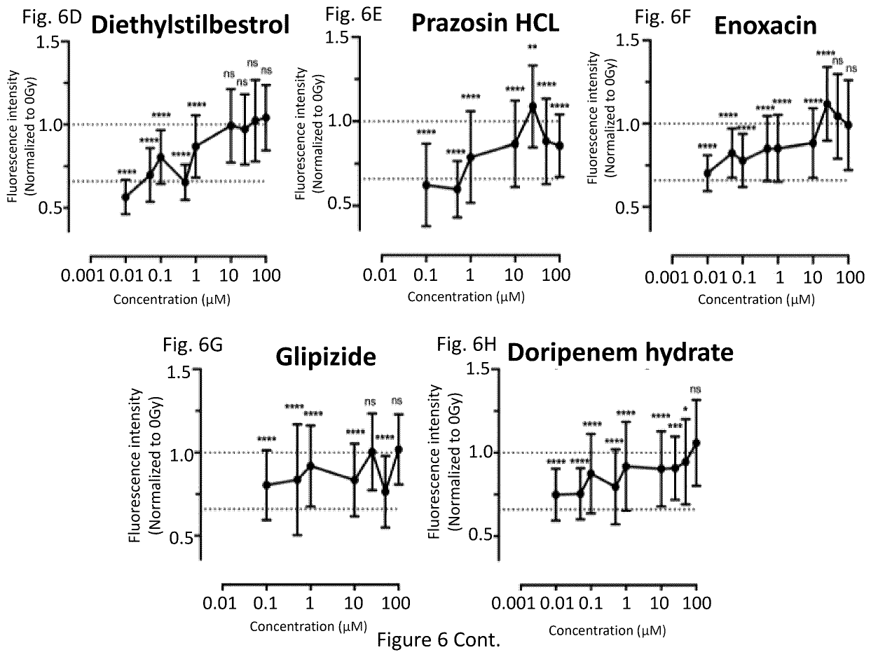

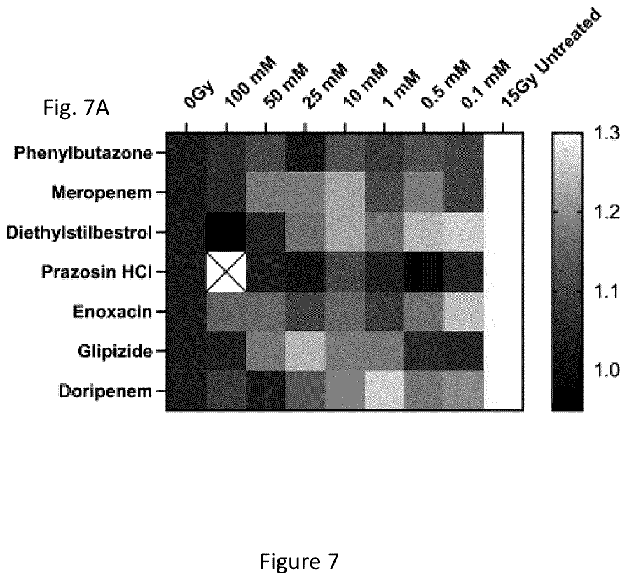

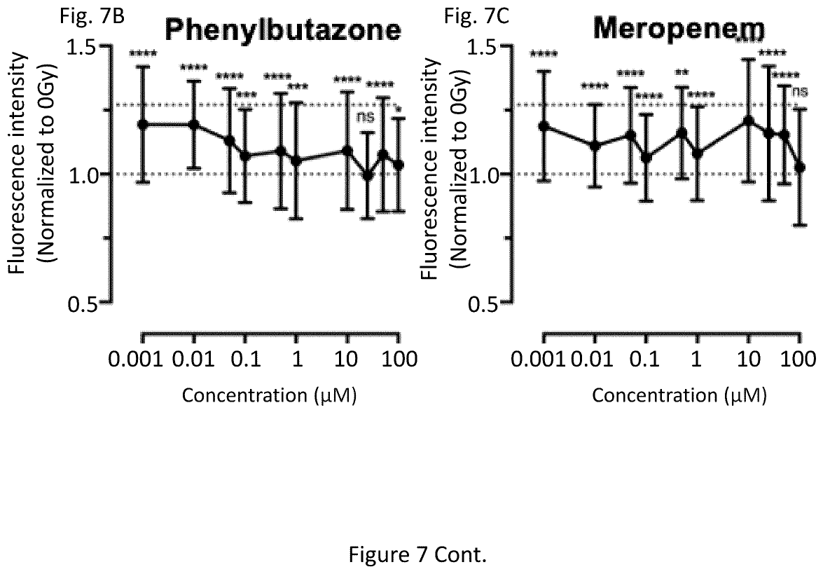

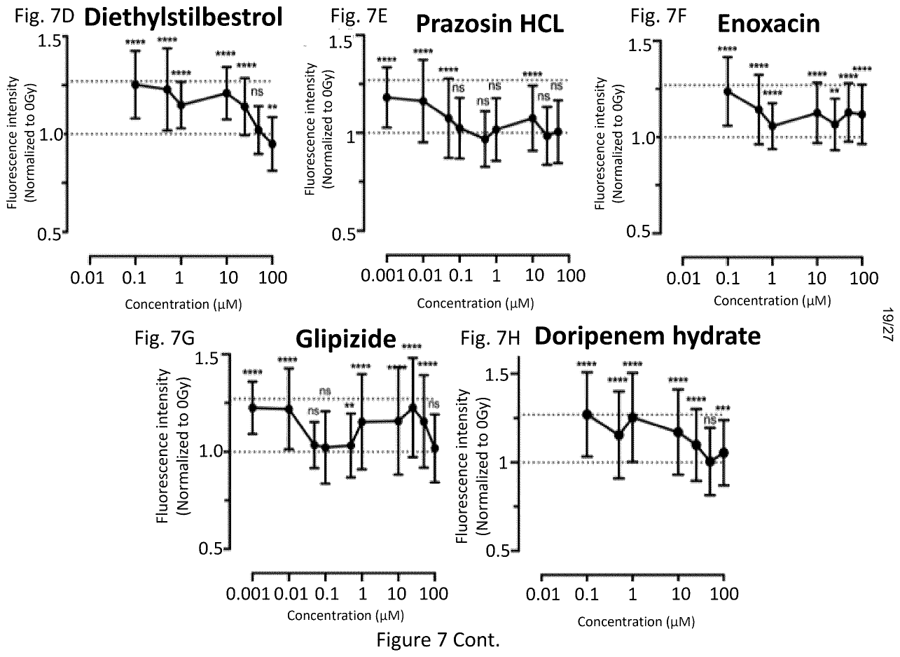

For the remaining 13 compounds, the drugs were tested for glutathione levels post-irradiation in dose-limiting experiments (1-100 μM) to identify effective concentrations. Many of the drugs were only effective at 100 μM and were excluded for lack of potency, leaving phenylbutazone, meropenem, diethylstilbestrol, prazosin, enoxacin, glipizide, and doripenem. Dose responses and heat maps summarizing the dose-response results for the glutathione and senescence assays are presented in FIG. 6 and FIG. 7, respectively. Dose-response curves for the glutathione assay show that phenylbutazone (FIG. 6B) and meropenem (FIG. 6C), exhibited radioprotection equivalent to 0 Gy over a concentration range 0.1-100 μM and Diethylstilbestrol (FIG. 6D) was radioprotective at 10-100 μM. Prazosin (FIG. 6E), Enoxacin (FIG. 6F), Glipizide (FIG. 6G), and Doripenem (FIG. 6H) showed protection only at higher concentrations.

The radioprotection trends based on the senescence assay differed somewhat, with phenylbutazone (FIG. 7B) and meropenem (FIG. 7C) showing only partial protection and Diethylstilbestrol (FIG. 7D) exhibiting radioprotection equivalent to 0 Gy between 50-100 μM concentrations whereas Prazosin (FIG. 7E) showed complete protection between 0.1-100 μM. Glipizide (FIG. 7G) and Doripenem (FIG. 7H) also showed variable protection.

EC₅₀ values extrapolated from dose response curves are shown in Table 4. Phenylbutazone showed the most promising results, with low EC₅₀ values for both the glutathione (0.08 μM) and senescence (0.05 μM) assays. Phenylbutazone is a non-steroidal anti-inflammatory drug (NSAID) that inhibits cyclooxygenases (COX-1 and COX-2), enzymes that produce prostaglandin (Lees, P et al., The Veterinary Journal 2013, 196, 294-303). Prostaglandins, specifically PGE2 signaling, have been shown to increase in irradiated salivary glands, and mitigation of salivary gland damage was achieved through treatment with the anti-inflammatory drug indomethacin (Jasmer, K J et al., J Clin Med 2020, 9, 4095; Gilman, K. E et al., Front Bioeng Biotechnol 2021, 9, 697671). Indomethacin also showed radioprotection in our drug screen but was ineffective at concentrations lower than 100 μM. Indomethacin only blocks COX-1 underscoring the greater efficacy of phenylbutazone.

TABLE 4
Estimated EC50 values estimated using nonlinear fit
in Prism for the top radioprotective drugs.

| | Estimated EC50 (μM) | |
|---|---|---|
| Compound | Glutathione | Senescence |
| Phenylbutazone | 0.08 | 0.05 |
| Meropenem | 0.1 | ND |
| Diethylstilbestrol | 0.7 | 37.7 |
| Prazosin HCl | 1 | 0.04 |
| Enoxacin | 2.4 | 0.5 |
| Glipizide | 4.0 | ND |
| Doripenem hydrate | 8.6 | 16.4 |

ND, not determined, Prism was unable to accurately fit a curve to the data.

Phenylbutazone was originally developed for chronic pain for conditions such as arthritis but has since been restricted to treatment of ankylosing spondylitis due to induction of rare but severe blood disorders, including anemia and leukopenia (Lees, P et al., The Veterinary Journal 2013, 196, 294-303). However, doses ranged from 300-1000 mg, generating a plasma concentration of 30-50 μg/mL. In contrast, an EC₅₀ of 0.08 μM for protecting against radiation-induced glutathione changes established in this invention equates to a concentration of 26 ng/mL. Thus, the risk of severe adverse effects may be greatly diminished for doses necessary for radioprotection. Additionally, phenylbutazone has excellent bioavailability (up to 90%) and a long half-life (50-105 hours), which may enable dose de-escalation further decreasing risks (Lees, P et al., The Veterinary Journal 2013, 196, 294-303; Smith, P et al., Xenobiotica 1987, 17, 435-443).

Enoxacin, an antibacterial agent used for treating urinary tract infections, has previously been identified as a radioprotective (Henwood, J et al., Drugs 1988, 36, 32-66). Using a high-throughput screening method with the viability of lymphocytes as the primary readout, two classes of antibiotics (tetracyclines and fluoroquinolones) were identified as robust radioprotectors, including enoxacin (Kim, K et al., Clin Cancer Res 2009, 15, 7238-7245). This observation matches with the screening results in this invention, in which several antibiotics were identified as double hits, including enoxacin, meropenem, doripenem hydrate, rifampin, and rifabutin. The Enoxacin EC₅₀ of 2.4 μM for the glutathione assay is similar to the 13 μM EC₅₀ reported for viability of lymphocyte cells (Kim, K et al., Clin Cancer Res 2009, 15, 7238-7245). Notably, five other fluoroquinolones reported as radioprotectors (levofloxacin, gatifloxacin, ofloxacin, moxifloxacin, and norfloxacin) were not hits in the present drug screen (Table 7) (Kim, K et al., Clin Cancer Res 2009, 15, 7238-7245). These disparities may be related to differences in cell type (salivary gland vs. lymphocyte) or readout (glutathione/senescence vs. viability).

Glipizide is a sulfonylurea, used to treat type 2 diabetes, promotes insulin secretion by binding to sulfonylurea receptor type 1 (SUR1), which closes ATP-sensitive potassium channels (Feingold, K. R, In Endotext; MDText.com, Inc: South Dartmouth, MA, 2000). The buildup of intracellular K⁺ causes membrane depolarization, opening voltage-gated calcium channels. Glyburide and gliclazide, similar sulfonylureas used as anti-diabetic medication, have been described as radioprotectors. Gliclazide was shown to have antioxidant activity, while glyburide was suggested to regulate apoptosis by controlling intracellular calcium and the mitochondrial permeability transition (MPT) pore (Pouri, M et al., Cardiovasc Hematol Agents Med Chem 2019, 17, 40-46, Epperly, M et al., Radioprotective Agents 2014; U.S. Pat. No. 8,883,852 B2).

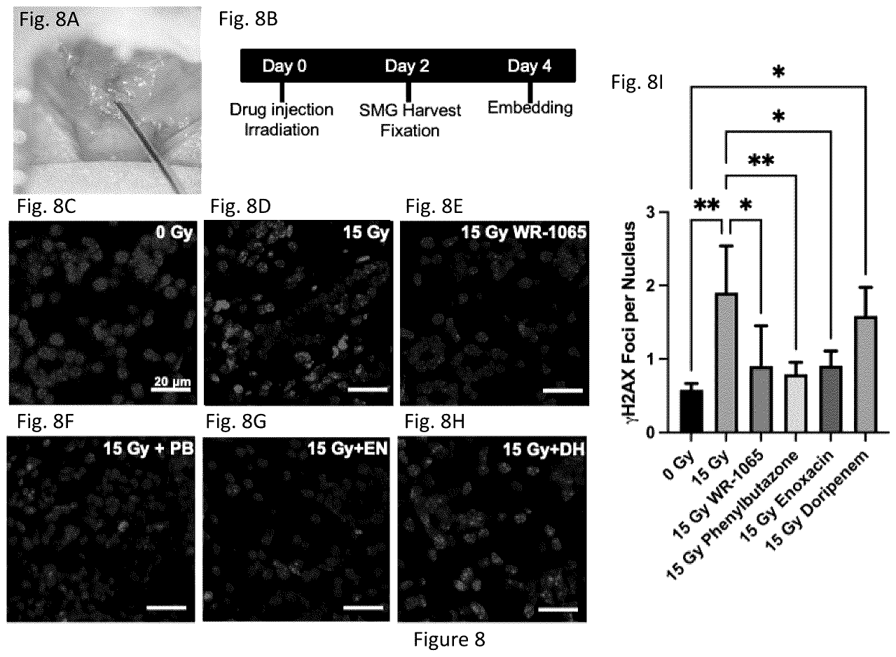

Based upon drug down selection data and measured EC₅₀ values, Phenylbutazone, Enoxacin, and Doripenem hydrate were selected for in vivo validation in mice with γH2AX foci per nucleus IHC staining as an outcome measure consistent with prior work showing correlation with the development of xerostomia (Song, Y. et al. Commun Biol 4, 361 (2021); Varghese, J. J. et al. J Vis Exp (2018); Varghese, J. J. et al. J Dent Res 97, 1252-1259 (2018); Varghese, J. J. et al. J Vis Exp (2018)). Vehicle-treated SGm exposed to 15 Gy radiation exhibited a 3.3-fold increase in the number of γH2AX foci per nucleus, indicating a significant increase in double-stranded DNA breaks due to radiation exposure (FIG. 8A through FIG. 8D and FIG. 8I). Treatment with WR-1065 resulted in a 0.5-fold reduction in γH2AX foci per nucleus compared to 15 Gy controls (FIG. 8D, FIG. 8E, and FIG. 8I). No significant differences existed between 0 Gy controls and WR-1065 treated SGm exposed to 15 Gy (FIG. 8C, FIG. 8E, and FIG. 8I). These results are similar to prior studies utilizing WR-1065 in vitro and in vivo via retrograde ductal injection (Varghese, J. J. et al. J Dent Res 97, 1252-1259 (2018); Song, Y. et al. Commun Biol 4, 361 (2021)). Treatment with the test compounds, Phenylbutazone, and Enoxacin resulted in 0.4- and 0.5-fold reduction in γH2AX foci per nucleus compared to 15 Gy controls, respectively (FIG. 8E through FIG. 8G and FIG. 8I). Results observed after Phenylbutazone treatment were not significantly different from 0 Gy controls (FIG. 8C, FIG. 8F, and FIG. 8I). Enoxacin treatment resulted in a 1.6-fold increase in γH2AX foci per nucleus (FIG. 8C, FIG. 8G, and FIG. 8I). Treatment with Doripenem hydrate did not reduce 7H2AX foci per nucleus relative to 15 Gy controls and showed a 2.7-fold increase compared to 0 Gy controls (FIG. 8C, FIG. 8D, FIG. 8H, and FIG. 8I).

The methods and materials employed are described herein.

### Materials

Detailed information on the drugs used for assay development and validation, including, Tempol, N-acetylcysteine, Edaravone, WR1065, Rapamycin, Ex-Rad, and Palifermin is listed in Table 5. Drug screening was completed using a 438 Selleck Chemicals library of FDA-approved drugs (Table 7). The screen's 25 top drug hits (Table 6) were purchased from Selleck Chemicals for dose-response studies and prepared and stored per manufacturer's instructions.

TABLE 5
Product information for radioprotective drugs for glutathione and
senescence assay validation.

| | | Catalog | | |
|---|---|---|---|---|
| Drug | Vendor | Number | Solvent | Storage |
| 4-hydroxy- | Sigma Aldrich | 176141 | DI Water | Stored at |
| TEMPO | | | | 4 °C; fresh |
| (Tempol) | | | | solution |
| | | | | mixed |
| | | | | each time |
| N- | Sigma Aldrich | A9165 | DI Water | Stored at |
| acetylcysteine | | | | 4 °C; fresh |
| | | | | solution |
| | | | | mixed |
| | | | | each time |
| 3-methyl- | Sigma Aldrich | M70800 | DMSO | Stored |
| 1-phenyl- | | | | at room |
| 2-pyrazoline- | | | | temp; fresh |
| 5-one | | | | solution |
| (Edaravone) | | | | mixed |
| | | | | each time |
| WR-1065 | Sigma Aldrich | W2020 | DI Water | −20 °C |
| Rapamycin | MedChemExpress | HY-10219 | DMSO | −80 °C |
| Ex-Rad | MedChemExpress | HY-101625 | DMSO | −80 °C |
| (Recilisib) | | | | |
| Keratinocyte | ThermoFisher | PHG00094 | PBS | −20 °C |
| Growth | (Invitrogen) | | | |
| Factor | | | | |
| (Palifermin) | | | | |

TABLE 6
Compounds identified as double hits in the drug screen.
Catalog numbers are from Selleck Chemicals

| Drug | Catalog Number |
|---|---|
| Adefovir Dipivoxil | S1718 |
| Albendazole | S1640 |
| Calcitriol | S1466 |
| Cilostazol | S1294 |
| Curcumin | S1848 |
| Didanosine | S1702 |
| Diethylstilbestrol | S1859 |
| Doripenem Hydrate | S1374 |
| Emtricitabine | S1704 |
| Enoxacin | S1756 |
| Eplerenone | S1707 |
| Etidronate | S1857 |
| Gemcitabine | S1714 |
| Glipizide | S1715 |
| Indomethacin | S1723 |
| Lomustine | S1840 |
| Melatonin | S1204 |
| Meropenem | S1381 |
| Nifedipine | S1808 |
| Oxcarbazepine | S1391 |
| Phenylbutazone | S1654 |
| Prazosin HC1 | S1424 |
| Rifabutin | S1741 |
| Rifampin | S1764 |
| Triamcinolone | S1933 |

TABLE 7
List of compounds tested from the Selleck Chemicals drug library. The normalized mean intensity,
standard deviation (STD), and p-values compared to 0 Gy are shown for each assay. Only drugs that
were nonsignificant compared to 0 Gy in the glutathione assay were tested with the senescence assay.

| | | Glutathione | | | Senescence | | |
|---|---|---|---|---|---|---|---|
| | | Normalized | | | Normalized | | |
| | | mean | | | mean | | |
| Compound | Cat # | intensity | STD | p-value | intensity | STD | p-value |
| Axitinib | S1005 | 0.7679 | 0.1917 | <0.0001 | | | |
| Bortezomib (Velcade) | S1013 | 0.2238 | 0.0136 | <0.0001 | | | |
| Bosutinib (SKI-606) | S1014 | 0.229 | 0.0219 | <0.0001 | | | |
| Dasatinib (BMS-354825) | S1021 | 0.7346 | 0.1086 | <0.0001 | | | |
| Erlotinib HCl | S1023 | 0.7755 | 0.0949 | <0.0001 | | | |
| Gefitinib (Iressa) | S1025 | 0.7189 | 0.132 | <0.0001 | | | |
| Imatinib Mesylate | S1026 | 0.6282 | 0.1543 | <0.0001 | | | |
| Lenalidomide | S1029 | 0.6051 | 0.1289 | <0.0001 | | | |
| Nilotinib (AMN-107) | S1033 | 0.6144 | 0.1278 | <0.0001 | | | |
| Rapamycin (Sirolimus) | S1039 | 0.9273 | 0.1972 | 0.2955 | | | |
| Sorafenib (Nexavar) | S1040 | 0.6416 | 0.1343 | <0.0001 | | | |
| Sunitinib Malate (Sutent) | S1042 | 0.2105 | 0.0177 | <0.0001 | | | |
| Temsirolimus (Torisel) | S1044 | 0.6431 | 0.1227 | <0.0001 | | | |
| Vandetanib (Zactima) | S1046 | 0.3433 | 0.0774 | <0.0001 | | | |
| Vorinostat (SAHA) | S1047 | 0.644 | 0.1435 | <0.0001 | | | |
| Masitinib (AB1010) | S1064 | 0.212 | 0.0226 | <0.0001 | | | |
| Crizotinib (PF-02341066) | S1068 | 0.1895 | 0.0274 | <0.0001 | | | |
| Vismodegib (GDC-0449) | S1082 | 0.9901 | 0.2133 | 0.9996 | 1.21 | 0.23 | <0.0001 |
| XL-184 (Cabozantinib) | S1119 | 0.5376 | 0.1178 | <0.0001 | | | |
| Everolimus (RAD001) | S1120 | 0.6322 | 0.1359 | <0.0001 | | | |
| Abiraterone (CB-7598) | S1123 | 0.7165 | 0.191 | <0.0001 | | | |
| Pemetrexed | S1135 | 0.5674 | 0.1686 | <0.0001 | | | |
| Malotilate | S1137 | 0.354 | 0.0912 | <0.0001 | | | |
| Ivacaftor (VX-770) | S1144 | 0.1563 | 0.0072 | <0.0001 | | | |
| Docetaxel (Taxotere) | S1148 | 0.6921 | 0.1702 | <0.0001 | | | |
| Paclitaxel (Taxol) | S1150 | 0.7346 | 0.1406 | <0.0001 | | | |
| Capecitabine (Xeloda) | S1156 | 0.8238 | 0.193 | <0.0001 | | | |
| Cisplatin | S1166 | 0.4557 | 0.103 | <0.0001 | | | |
| Valproic acid sodium salt | S1168 | 0.4158 | 0.0911 | <0.0001 | | | |
| (Sodium valproate) | | | | | | | |
| Regorafenib (BAY 73-4506) | S1178 | 0.8772 | 0.1611 | 0.0031 | | | |
| Ritonavir | S1185 | 0.63 | 0.13 | <0.0001 | | | |
| Anastrozole | S1188 | 0.7402 | 0.1621 | <0.0001 | | | |
| Aprepitant (MK-0869) | S1189 | 0.6059 | 0.1491 | <0.0001 | | | |
| Bicalutamide (Casodex) | S1190 | 0.2862 | 0.0504 | <0.0001 | | | |
| Fulvestrant (Faslodex) | S1191 | 0.2869 | 0.0684 | <0.0001 | | | |
| Thalidomide | S1193 | 0.2465 | 0.0238 | <0.0001 | | | |
| Exemestane | S1196 | 0.5404 | 0.1112 | <0.0001 | | | |
| Finasteride | S1197 | 0.5653 | 0.1251 | <0.0001 | | | |
| Irinotecan | S1198 | 0.4949 | 0.0971 | <0.0001 | | | |
| Cladribine | S1199 | 0.649 | 0.1529 | <0.0001 | | | |
| Decitabine | S1200 | 0.5066 | 0.0977 | <0.0001 | | | |
| Dutasteride | S1202 | 0.788 | 0.1763 | <0.0001 | | | |
| Melatonin | S1204 | 0.9402 | 0.2157 | 0.2337 | 1.056 | 0.15 | 0.0587 |
| Bisoprolol | S1206 | 0.8501 | 0.1525 | <0.0001 | | | |
| Doxorubicin (Adriamycin) | S1208 | 0.4119 | 0.0514 | <0.0001 | | | |
| Adrucil (Fluorouracil) | S1209 | 0.9115 | 0.2002 | 0.0115 | | | |
| Abitrexate (Methotrexate) | S1210 | 0.5385 | 0.0896 | <0.0001 | | | |
| Imiquimod | S1211 | 0.347 | 0.0676 | <0.0001 | | | |
| Bendamustine HCL | S1212 | 0.6456 | 0.136 | <0.0001 | | | |
| Nelarabine (Arranon) | S1213 | 0.4648 | 0.1077 | <0.0001 | | | |
| Bleomycin sulfate | S1214 | 0.7651 | 0.1153 | <0.0001 | | | |
| Carboplatin | S1215 | 0.4303 | 0.0992 | <0.0001 | | | |
| Clafen (Cyclophosphamide) | S1217 | 0.5778 | 0.1129 | <0.0001 | | | |
| Clofarabine | S1218 | 0.2138 | 0.0267 | <0.0001 | | | |
| Dacarbazine (DTIC-Dome) | S1221 | 0.4927 | 0.0907 | <0.0001 | | | |
| Dexrazoxane | S1222 | 0.7489 | 0.1336 | <0.0001 | | | |
| Hydrochloride | | | | | | | |
| Epirubicin Hydrochloride | S1223 | 0.182 | 0.0395 | <0.0001 | | | |
| Oxaliplatin (Eloxatin) | S1224 | 0.4691 | 0.1073 | <0.0001 | | | |
| Etoposide (VP-16) | S1225 | 0.4605 | 0.1029 | <0.0001 | | | |
| Evista (Raloxifene | S1227 | 0.1288 | 0.0083 | <0.0001 | | | |
| Hydrochloride) | | | | | | | |
| Idarubicin HCl | S1228 | 0.1332 | 0.0065 | <0.0001 | | | |
| Topotecan HCl | S1231 | 0.237 | 0.0285 | <0.0001 | | | |
| 2-Methoxyestradiol | S1233 | 0.2997 | 0.0531 | <0.0001 | | | |
| Letrozole | S1235 | 0.6447 | 0.139 | <0.0001 | | | |
| Leucovorin Calcium | S1236 | 0.6894 | 0.1596 | <0.0001 | | | |
| Methazolastone | S1237 | 0.7841 | 0.1687 | <0.0001 | | | |
| Vincristine | S1241 | 0.6827 | 0.1199 | <0.0001 | | | |
| Agomelatine | S1243 | 0.545 | 0.108 | <0.0001 | | | |
| Leflunomide | S1247 | 0.4705 | 0.0838 | <0.0001 | | | |
| Vinblastine | S1248 | 0.4679 | 0.0922 | <0.0001 | | | |
| MDV3100 (Enzalutamide) | S1250 | 0.5927 | 0.1491 | <0.0001 | | | |
| Dienogest | S1251 | 0.6917 | 0.1508 | <0.0001 | | | |
| Entecavir hydrate | S1252 | 0.2629 | 0.0421 | <0.0001 | | | |
| Nepafenac | S1255 | 0.3547 | 0.0835 | <0.0001 | | | |
| Rufinamide (Banzel) | S1256 | 0.3032 | 0.0687 | <0.0001 | | | |
| Posaconazole | S1257 | 0.6714 | 0.122 | <0.0001 | | | |
| Prasugrel (Effient) | S1258 | 0.5374 | 0.1287 | <0.0001 | | | |
| Ramelteon (TAK-375) | S1259 | 0.7926 | 0.1877 | <0.0001 | | | |
| AMG-073 HCl | S1260 | 0.7137 | 0.1449 | <0.0001 | | | |
| (Cinacalcet hydrochloride) | | | | | | | |
| Celecoxib | S1261 | 0.6671 | 0.1584 | <0.0001 | | | |
| Vemurafenib (PLX4032) | S1267 | 0.8184 | 0.1417 | <0.0001 | | | |
| Acarbose | S1271 | 0.6125 | 0.1194 | <0.0001 | | | |
| Adapalene | S1276 | 0.7821 | 0.1635 | <0.0001 | | | |
| Altretamine (Hexalen) | S1278 | 0.8938 | 0.1409 | 0.0009 | | | |
| Amisulpride | S1280 | 0.7907 | 0.219 | <0.0001 | | | |
| Aniracetam | S1281 | 0.9526 | 0.2748 | 0.5968 | 1.099 | 0.08 | <0.0001 |
| Artemisinin | S1282 | 0.5704 | 0.1297 | <0.0001 | | | |
| Asenapine | S1283 | 0.3495 | 0.0651 | <0.0001 | | | |
| Benazepril hydrochloride | S1284 | 0.8483 | 0.1974 | <0.0001 | | | |
| Biperiden HCl | S1285 | 0.7237 | 0.1717 | <0.0001 | | | |
| Budesonide | S1286 | 0.6944 | 0.1369 | <0.0001 | | | |
| Bumetanide | S1287 | 0.7638 | 0.1665 | <0.0001 | | | |
| Camptothecin | S1288 | 0.6391 | 0.0694 | <0.0001 | | | |
| Carmofur | S1289 | 0.681 | 0.1593 | <0.0001 | | | |
| Cetirizine | S1291 | 0.724 | 0.1549 | <0.0001 | | | |
| Dihydrochloride | | | | | | | |
| Cilnidipine | S1293 | 0.7279 | 0.1093 | <0.0001 | | | |
| Cilostazol | S1294 | 0.9114 | 0.1338 | 0.0706 | 1.015 | 0.11 | 0.6189 |
| Floxuridine | S1299 | 0.7282 | 0.1457 | <0.0001 | | | |
| Ftorafur | S1300 | 0.8765 | 0.1331 | 0.0066 | | | |
| Ifosfamide | S1302 | 0.7162 | 0.1141 | <0.0001 | | | |
| Megestrol Acetate | S1304 | 0.7336 | 0.1658 | <0.0001 | | | |
| Mercaptopurine | S1305 | 0.706 | 0.1466 | <0.0001 | | | |
| Pamidronate Disodium | S1311 | 0.7776 | 0.1403 | <0.0001 | | | |
| Streptozotocin (Zanosar) | S1312 | 0.8466 | 0.1613 | 0.0001 | | | |
| Zoledronic Acid | S1314 | 0.7686 | 0.1155 | <0.0001 | | | |
| (Zoledronate) | | | | | | | |
| Dexamethasone | S1322 | 0.7705 | 0.1934 | <0.0001 | | | |
| Doxazosin mesylate | S1324 | 0.7306 | 0.1484 | <0.0001 | | | |
| Edaravone (MCI-186) | S1326 | 0.7096 | 0.1208 | <0.0001 | | | |
| Ellagic acid | S1327 | 0.5742 | 0.1173 | <0.0001 | | | |
| Etodolac (Lodine) | S1328 | 0.6429 | 0.1226 | <0.0001 | | | |
| Etomidate | S1329 | 0.764 | 0.1769 | <0.0001 | | | |
| Felbamate | S1330 | 0.486 | 0.0868 | <0.0001 | | | |
| Fluconazole | S1331 | 0.3299 | 0.045 | <0.0001 | | | |
| Flumazenil | S1332 | 0.3998 | 0.0809 | <0.0001 | | | |
| Fluoxetine HCl | S1333 | 0.5514 | 0.1137 | <0.0001 | | | |
| Fluvoxamine maleate | S1336 | 0.7316 | 0.1388 | <0.0001 | | | |
| Gatifloxacin | S1340 | 0.7042 | 0.1547 | <0.0001 | | | |
| Genistein | S1342 | 0.4313 | 0.0827 | <0.0001 | | | |
| Glimepiride | S1344 | 0.5175 | 0.1035 | <0.0001 | | | |
| Granisetron HCl | S1345 | 0.6701 | 0.1365 | <0.0001 | | | |
| Ivermectin | S1351 | 0.3362 | 0.0604 | <0.0001 | | | |
| Ketoconazole | S1353 | 0.6026 | 0.1116 | <0.0001 | | | |
| Lansoprazole | S1354 | 0.6174 | 0.1145 | <0.0001 | | | |
| Levetiracetam | S1356 | 0.8578 | 0.1527 | <0.0001 | | | |
| Lidocaine (Alphacaine) | S1357 | 0.8421 | 0.1326 | <0.0001 | | | |
| Loratadine | S1358 | 0.6211 | 0.1116 | <0.0001 | | | |
| Acitretin | S1368 | 0.4424 | 0.0842 | <0.0001 | | | |
| Biapenem | S1370 | 0.7155 | 0.1158 | <0.0001 | | | |
| Cefoselis sulfate | S1371 | 1.093 | 0.3285 | 0.2906 | 1.158 | 0.11 | <0.0001 |
| Daptomycin | S1373 | 1.162 | 0.2819 | 0.0006 | 1.379 | 0.28 | <0.0001 |
| Doripenem Hydrate | S1374 | 0.9587 | 0.2554 | 0.9982 | 1.047 | 0.29 | 0.481 |
| Dorzolamide HCL | S1375 | 0.667 | 0.2138 | <0.0001 | | | |
| Gestodene | S1376 | 0.5871 | 0.1663 | <0.0001 | | | |
| Drospirenone | S1377 | 0.5756 | 0.1338 | <0.0001 | | | |
| Ruxolitinib | S1378 | 0.7046 | 0.1915 | <0.0001 | | | |
| (INCB018424) | | | | | | | |
| Isotretinoin | S1379 | 0.9241 | 0.2119 | 0.5325 | 1.256 | 0.29 | <0.0001 |
| Lopinavir (ABT-378) | S1380 | 0.6059 | 0.139 | <0.0001 | | | |
| Meropenem | S1381 | 0.9382 | 0.2223 | 0.8517 | 1.048 | 0.19 | 0.5334 |
| Mianserin hydrochloride | S1382 | 0.619 | 0.1365 | <0.0001 | | | |
| Minoxidil | S1383 | 0.7049 | 0.1728 | <0.0001 | | | |
| Mosapride citrate | S1385 | 0.7418 | 0.2162 | <0.0001 | | | |
| Nafamostat mesylate | S1386 | 0.7493 | 0.1488 | <0.0001 | | | |
| Omeprazole (Prilosec) | S1389 | 0.9084 | 0.2218 | 0.2776 | 1.16 | 0.15 | <0.0001 |
| Oxcarbazepine | S1391 | 1.062 | 0.2542 | 0.819 | 0.9502 | 0.14 | 0.717 |
| Pizotifen malate | S1394 | 0.8876 | 0.1745 | 0.0959 | 1.329 | 0.26 | <0.0001 |
| Resveratrol | S1396 | 0.6659 | 0.2095 | <0.0001 | | | |
| Rocuronium bromide | S1397 | 0.8135 | 0.1979 | <0.0001 | | | |
| Stavudine | S1398 | 0.6179 | 0.1966 | <0.0001 | | | |
| Teicoplanin | S1399 | 0.8745 | 0.2054 | 0.0323 | 1.348 | 0.25 | <0.0001 |
| Tenofovir (Viread) | S1401 | 0.8735 | 0.2555 | 0.0451 | 1.106 | 0.29 | 0.0011 |
| Tigecycline | S1403 | 0.6601 | 0.1346 | <0.0001 | | | |
| Trilostane | S1404 | 0.6923 | 0.1892 | <0.0001 | | | |
| Vecuronium Bromide | S1405 | 0.55 | 0.1319 | <0.0001 | | | |
| Bimatoprost | S1407 | 0.717 | 0.1918 | <0.0001 | | | |
| Linezolid (Zyvox) | S1408 | 0.6209 | 0.1736 | <0.0001 | | | |
| Alfuzosin hydrochloride | S1409 | 0.7318 | 0.2139 | <0.0001 | | | |
| (Uroxatral) | | | | | | | |
| Clopidogrel (Plavix) | S1415 | 0.7618 | 0.2116 | <0.0001 | | | |
| Prazosin HCl | S1424 | 1.095 | 0.2835 | 0.2007 | 0.9174 | 0.08 | 0.1369 |
| Repaglinide | S1426 | 0.6726 | 0.1873 | <0.0001 | | | |
| Risedronate sodium | S1428 | 0.6486 | 0.1807 | <0.0001 | | | |
| Rolipram | S1430 | 0.7034 | 0.1988 | <0.0001 | | | |
| Sildenafil citrate | S1431 | 0.662 | 0.1344 | <0.0001 | | | |
| Sumatriptan succinate | S1432 | 0.8677 | 0.2307 | 0.0293 | 1.416 | 0.24 | <0.0001 |
| Tianeptine sodium | S1436 | 0.6 | 0.1785 | <0.0001 | | | |
| Tizanidine HCl | S1437 | 0.7164 | 0.2287 | <0.0001 | | | |
| Topiramate | S1438 | 0.776 | 0.2183 | <0.0001 | | | |
| Tranilast (SB 252218) | S1439 | 0.7429 | 0.1807 | <0.0001 | | | |
| Varenicline tartrate | S1440 | 0.2934 | 0.0376 | <0.0001 | | | |
| Venlafaxine | S1441 | 0.4823 | 0.1332 | <0.0001 | | | |
| Voriconazole | S1442 | 0.5288 | 0.1418 | <0.0001 | | | |
| Zileuton | S1443 | 0.4027 | 0.1007 | <0.0001 | | | |
| Ziprasidone hydrochloride | S1444 | 0.2477 | 0.0317 | <0.0001 | | | |
| Zonisamide | S1445 | 0.5928 | 0.1402 | <0.0001 | | | |
| Atazanavir sulfate | S1457 | 0.6514 | 0.1379 | <0.0001 | | | |
| Ofloxacin (Floxin) | S1463 | 0.7141 | 0.1632 | <0.0001 | | | |
| Marbofloxacin | S1464 | 0.7546 | 0.1816 | <0.0001 | | | |
| Moxifloxacin | S1465 | 0.5719 | 0.1285 | <0.0001 | | | |
| hydrochloride | | | | | | | |
| Calcitriol (Rocaltrol) | S1466 | 0.9747 | 0.2419 | 0.9987 | 0.9927 | 0.09 | 0.9993 |
| Doxercalciferol (Hectorol) | S1467 | 0.6828 | 0.1696 | <0.0001 | | | |
| Alfacalcidol | S1468 | 0.6596 | 0.1745 | <0.0001 | | | |
| Calcifediol | S1469 | 0.2451 | 0.0504 | <0.0001 | | | |
| Iloperidone (Fanapt) | S1483 | 0.3466 | 0.0792 | <0.0001 | | | |
| Naratriptan HCl | S1488 | 0.3634 | 0.0806 | <0.0001 | | | |
| Ponatinib (AP24534) | S1490 | 0.5698 | 0.0444 | <0.0001 | | | |
| Fludarabine (Fludara) | S1491 | 0.2976 | 0.0599 | <0.0001 | | | |
| Pralatrexate (Folotyn) | S1497 | 0.6784 | 0.1651 | <0.0001 | | | |
| Cefaclor (Ceclor) | S1499 | 0.6233 | 0.1376 | <0.0001 | | | |
| Betamethasone | S1500 | 0.4986 | 0.1044 | <0.0001 | | | |
| (Celestone) | | | | | | | |
| Mycophenolate mofetil | S1501 | 0.3232 | 0.0606 | <0.0001 | | | |
| (CellCept) | | | | | | | |
| Cephalexin (Cefalexin) | S1502 | 0.6251 | 0.1367 | <0.0001 | | | |
| Dyphylline (Dilor) | S1504 | 0.8217 | 0.1517 | <0.0001 | | | |
| Aztreonam (Azactam, | S1505 | 0.8347 | 0.1959 | <0.0001 | | | |
| Cayston) | | | | | | | |
| Perindopril Erbumine | S1506 | 0.2136 | 0.0211 | <0.0001 | | | |
| (Aceon) | | | | | | | |
| Irbesartan (Avapro) | S1507 | 0.38 | 0.0691 | <0.0001 | | | |
| Alprostadil (Caverject) | S1508 | 0.4821 | 0.1049 | <0.0001 | | | |
| Norfloxacin (Norxacin) | S1509 | 0.569 | 0.1354 | <0.0001 | | | |
| Tadalafil (Cialis) | S1512 | 0.6341 | 0.1598 | <0.0001 | | | |
| Cyclosporine (Neoral) | S1514 | 0.1675 | 0.0202 | <0.0001 | | | |
| Cidofovir (Vistide) | S1516 | 0.3992 | 0.0836 | <0.0001 | | | |
| Natamycin (Pimaricin) | S1517 | 0.5218 | 0.1167 | <0.0001 | | | |
| Vinorelbine (Navelbine) | S1527 | 0.6536 | 0.1233 | <0.0001 | | | |
| Telaprevir (VX-950) | S1538 | 0.382 | 0.087 | <0.0001 | | | |
| Saxagliptin (BMS-477118, | S1540 | 0.8092 | 0.1763 | <0.0001 | | | |
| Onglyza) | | | | | | | |
| Febuxostat (Uloric) | S1547 | 0.6091 | 0.1466 | <0.0001 | | | |
| Nebivolol (Bystolic) | S1549 | 0.1482 | 0.0387 | <0.0001 | | | |
| Pimobendan (Vetmedin) | S1550 | 0.3234 | 0.0734 | <0.0001 | | | |
| Pomalidomide | S1567 | 0.2136 | 0.0357 | <0.0001 | | | |
| Tazarotene (Avage) | S1569 | 0.3553 | 0.0378 | <0.0001 | | | |
| Sulfasalazine (Azulfidine) | S1576 | 0.5411 | 0.1266 | <0.0001 | | | |
| Candesartan (Atacand) | S1578 | 0.7132 | 0.1392 | <0.0001 | | | |
| Ubenimex (Bestatin) | S1591 | 0.5641 | 0.1318 | <0.0001 | | | |
| Apixaban | S1593 | 0.5027 | 0.1044 | <0.0001 | | | |
| Reserpine | S1601 | 0.2871 | 0.0393 | <0.0001 | | | |
| Furosemide (Lasix) | S1603 | 0.3911 | 0.098 | <0.0001 | | | |
| Olmesartan medoxomil | S1604 | 0.5219 | 0.1216 | <0.0001 | | | |
| (Benicar) | | | | | | | |
| Cefdinir (Omnicef) | S1605 | 0.5287 | 0.1625 | <0.0001 | | | |
| Clotrimazole (Canesten) | S1606 | 0.2715 | 0.0658 | <0.0001 | | | |
| Rizatriptan Benzoate | S1607 | 0.1881 | 0.0328 | <0.0001 | | | |
| (Maxalt) | | | | | | | |
| Pyridostigmine Bromide | S1608 | 0.3614 | 0.0727 | <0.0001 | | | |
| (Mestinon) | | | | | | | |
| Methimazole | S1609 | 0.2442 | 0.042 | <0.0001 | | | |
| (Tapazole, Northyx) | | | | | | | |
| Metolazone (Zaroxolyn) | S1610 | 0.263 | 0.0438 | <0.0001 | | | |
| Cefoperazone (Cefobid) | S1611 | 0.5032 | 0.1136 | <0.0001 | | | |
| Silodosin (Rapaflo) | S1613 | 0.4797 | 0.1169 | <0.0001 | | | |
| Riluzole (Rilutek) | S1614 | 0.5553 | 0.151 | <0.0001 | | | |
| Risperidone (Risperdal) | S1615 | 0.5694 | 0.1427 | <0.0001 | | | |
| Sulfapyridine (Dagenan) | S1617 | 0.6552 | 0.1223 | <0.0001 | | | |
| Sulfameter (Bayrena) | S1618 | 0.7405 | 0.1721 | <0.0001 | | | |
| Prilocaine | S1619 | 0.6334 | 0.1346 | <0.0001 | | | |
| Darunavir Ethanolate | S1620 | 0.6248 | 0.153 | <0.0001 | | | |
| (Prezista) | | | | | | | |
| Prednisone (Adasone) | S1622 | 0.5534 | 0.1191 | <0.0001 | | | |
| Acetylcysteine | S1623 | 0.5432 | 0.1166 | <0.0001 | | | |
| Alendronate (Fosamax) | S1624 | 0.7395 | 0.1868 | <0.0001 | | | |
| Ethinyl Estradiol | S1625 | 0.5856 | 0.1325 | <0.0001 | | | |
| Naproxen (Aleve) | S1626 | 0.6014 | 0.1291 | <0.0001 | | | |
| Nitazoxanide | S1627 | 0.5996 | 0.1537 | <0.0001 | | | |
| (Alinia, Annita) | | | | | | | |
| Orlistat (Alli, Xenical) | S1629 | 0.5993 | 0.1425 | <0.0001 | | | |
| Allopurinol (Zyloprim) | S1630 | 0.6143 | 0.1782 | <0.0001 | | | |
| Zafirlukast (Accolate) | S1633 | 0.3923 | 0.0732 | <0.0001 | | | |
| Erythromycin (E-Mycin) | S1635 | 0.5286 | 0.1569 | <0.0001 | | | |
| Amphotericin B (Abelcet) | S1636 | 0.4996 | 0.0869 | <0.0001 | | | |
| Docosanol (Abreva) | S1637 | 0.8771 | 0.2199 | 0.0737 | 1.125 | 0.12 | <0.0001 |
| Ibuprofen (Advil) | S1638 | 0.8226 | 0.2278 | 0.0019 | | | |
| Amprenavir (Agenerase) | S1639 | 0.6951 | 0.1728 | <0.0001 | | | |
| Albendazole (Albenza) | S1640 | 0.8968 | 0.1932 | 0.2657 | 0.9835 | 0.07 | 0.9561 |
| Chlorothiazide | S1641 | 0.9869 | 0.2016 | 0.9996 | 1.077 | 0.07 | <0.0001 |
| Methyldopa (Aldomet) | S1642 | 0.7124 | 0.2191 | <0.0001 | | | |
| Ursodiol (Actigal Urso) | S1643 | 0.947 | 0.1761 | 0.9671 | 1.069 | 0.12 | <0.0001 |
| Nitrofurazone (Nitrofural) | S1644 | 0.3224 | 0.0826 | <0.0001 | | | |
| Ketoprofen (Actron) | S1645 | 0.8417 | 0.2378 | 0.0009 | | | |
| Ketorolac (Toradol) | S1646 | 0.5049 | 0.1605 | <0.0001 | | | |
| Adenosine (Adenocard) | S1647 | 0.7585 | 0.1942 | <0.0001 | | | |
| Cytarabine | S1648 | 0.6111 | 0.163 | <0.0001 | | | |
| Zolmitriptan (Zomig) | S1649 | 0.5816 | 0.1721 | <0.0001 | | | |
| Telbivudine | S1651 | 0.5754 | 0.1379 | <0.0001 | | | |
| (Sebivo, Tyzeka) | | | | | | | |
| Monobenzone (Benoquin) | S1652 | 0.458 | 0.1088 | <0.0001 | | | |
| Tretinoin (Aberela) | S1653 | 0.4902 | 0.1183 | <0.0001 | | | |
| Phenylbutazone | S1654 | 1.01 | 0.2566 | 0.9997 | 1.037 | 0.09 | 0.1341 |
| (Butazolidin, Butatron) | | | | | | | |
| Ezetimibe (Zetia) | S1655 | 0.6562 | 0.2066 | <0.0001 | | | |
| Enalaprilat dihydrate | S1657 | 0.7903 | 0.2444 | <0.0001 | | | |
| Dofetilide (Tikosyn) | S1658 | 0.3721 | 0.0907 | <0.0001 | | | |
| Isradipine (Dynacirc) | S1662 | 0.6919 | 0.1531 | <0.0001 | | | |
| Estrone | S1665 | 0.6293 | 0.1749 | <0.0001 | | | |
| Flucytosine (Ancobon) | S1666 | 0.758 | 0.2262 | <0.0001 | | | |
| Trichlormethiazide | S1667 | 1.027 | 0.3153 | 0.9989 | 1.015 | 0.1 | 0.951 |
| (Achletin) | | | | | | | |
| Loteprednol etabonate | S1669 | 0.5606 | 0.1401 | <0.0001 | | | |
| Aminocaproic acid | S1671 | 0.6283 | 0.1727 | <0.0001 | | | |
| (Amicar) | | | | | | | |
| Aminoglutethimide | S1672 | 0.4593 | 0.1122 | <0.0001 | | | |
| (Cytadren) | | | | | | | |
| Aminophylline | S1673 | 0.5803 | 0.1587 | <0.0001 | | | |
| (Truphylline) | | | | | | | |
| Amorolfine Hydrochloride | S1676 | 0.614 | 0.1705 | <0.0001 | | | |
| Chloramphenicol | S1677 | 0.8165 | 0.2027 | 0.0001 | | | |
| (Chloromycetin) | | | | | | | |
| Flurbiprofen (Ansaid) | S1679 | 0.5051 | 0.1549 | <0.0001 | | | |
| Disulfiram (Antabuse) | S1680 | 0.1133 | 0.009 | <0.0001 | | | |
| Mesalamine (Lialda) | S1681 | 0.392 | 0.142 | <0.0001 | | | |
| Ipratropium bromide | S1683 | 0.2741 | 0.0914 | <0.0001 | | | |
| Sulfanilamide | S1685 | 0.3122 | 0.1237 | <0.0001 | | | |
| Betapar (Meprednisone) | S1689 | 0.2137 | 0.1127 | <0.0001 | | | |
| Praziquantel (Biltricide) | S1691 | 0.7221 | 0.1669 | <0.0001 | | | |
| Busulfan | S1692 | 0.9131 | 0.2068 | 0.4031 | 1.046 | 0.1 | 0.0238 |
| (Myleran, Busulfex) | | | | | | | |
| Carbamazepine | S1693 | 0.829 | 0.1809 | 0.0027 | | | |
| (Carbatrol) | | | | | | | |
| Hydrocortisone (Cortisol) | S1696 | 0.8679 | 0.2371 | 0.0362 | | | |
| Torsemide (Demadex) | S1698 | 0.775 | 0.2033 | <0.0001 | | | |
| Desonide | S1701 | 0.8074 | 0.1824 | 0.0002 | | | |
| Didanosine (Videx) | S1702 | 0.955 | 0.1902 | 0.9872 | 0.9957 | 0.09 | 0.9996 |
| Divalproex sodium | S1703 | 0.6238 | 0.1103 | <0.0001 | | | |
| Emtricitabine (Emtriva) | S1704 | 0.8965 | 0.2104 | 0.2221 | 1.036 | 0.09 | 0.1164 |
| Progesterone | S1705 | 0.7941 | 0.1989 | <0.0001 | | | |
| (Prometrium) | | | | | | | |
| Lamivudine (Epivir) | S1706 | 0.2214 | 0.0329 | <0.0001 | | | |
| Eplerenone | S1707 | 0.908 | 0.2475 | 0.4024 | 1.003 | 0.11 | 0.9997 |
| Hydrochlorothiazide | S1708 | 0.4922 | 0.101 | <0.0001 | | | |
| Estradiol | S1709 | 0.9775 | 0.1907 | 0.9993 | | | |
| Deferasirox (Exjade) | S1712 | 0.8192 | 0.1973 | 0.0013 | | | |
| Piroxicam (Feldene) | S1713 | 0.8729 | 0.2166 | 0.0459 | | | |
| Gemcitabine (Gemzar) | S1714 | 0.9066 | 0.2114 | 0.3055 | 1.037 | 0.09 | 0.1097 |
| Glipizide (Glucotrol) | S1715 | 0.925 | 0.1851 | 0.7089 | 0.9953 | 0.17 | 0.9996 |
| Glyburide (Diabeta) | S1716 | 0.7705 | 0.1888 | <0.0001 | | | |
| Adefovir Dipivoxil | S1718 | 0.9028 | 0.167 | 0.2793 | 0.9738 | 0.06 | 0.3965 |
| (Preveon, Hepsera) | | | | | | | |
| Zalcitabine | S1719 | 0.8142 | 0.1743 | 0.0006 | | | |
| Azathioprine | S1721 | 0.833 | 0.151 | 0.0068 | | | |
| (Azasan, Imuran) | | | | | | | |
| Indomethacin | S1723 | 0.9533 | 0.2145 | 0.9936 | 0.9666 | 0.1 | 0.1851 |
| (Indocid, Indocin) | | | | | | | |
| Paliperidone (Invega) | S1724 | 0.816 | 0.1543 | 0.001 | | | |
| Terbinafine | S1725 | 0.5309 | 0.1007 | <0.0001 | | | |
| (Lamisil, Terbinex) | | | | | | | |
| Levodopa (Sinemet) | S1726 | 0.7236 | 0.2429 | <0.0001 | | | |
| Levonorgestrel | S1727 | 0.5853 | 0.2156 | <0.0001 | | | |
| (Levonelle) | | | | | | | |
| Gemfibrozil (Lopid) | S1729 | 1.001 | 0.2817 | >0.9999 | 1.134 | 0.21 | <0.0001 |
| Indapamide (Lozol) | S1730 | 0.8668 | 0.2296 | 0.0893 | 1.158 | 0.19 | <0.0001 |
| Mitotane (Lysodren) | S1732 | 0.8592 | 0.1914 | 0.0506 | 1.345 | 0.25 | <0.0001 |
| Methylprednisolone | S1733 | 0.8076 | 0.1802 | 0.0007 | | | |
| Meloxicam (Mobic) | S1734 | 0.7418 | 0.1984 | <0.0001 | | | |
| Mesna | S1735 | 0.7905 | 0.1789 | 0.0002 | | | |
| (Uromitexan, Mesnex) | | | | | | | |
| Methocarbamol (Robaxin) | S1736 | 0.3033 | 0.0711 | <0.0001 | | | |
| Prednisolone | S1737 | 0.9768 | 0.2346 | 0.9994 | 1.139 | 0.23 | <0.0001 |
| (Hydroretrocortine) | | | | | | | |
| Telmisartan (Micardis) | S1738 | 0.8437 | 0.2253 | 0.0265 | | | |
| Thiabendazole | S1739 | 0.6245 | 0.197 | <0.0001 | | | |
| Guaifenesin | S1740 | 0.5289 | 0.1281 | <0.0001 | | | |
| (Guaiphenesin) | | | | | | | |
| Rifabutin (Mycobutin) | S1741 | 0.897 | 0.2248 | 0.2976 | 1.045 | 0.14 | 0.1788 |
| Nevirapine (Viramune) | S1742 | 0.849 | 0.171 | 0.0206 | | | |
| Niacin (Nicotinic acid) | S1744 | 0.6951 | 0.2021 | <0.0001 | | | |
| Nimodipine (Nimotop) | S1747 | 0.6417 | 0.2019 | <0.0001 | | | |
| Nisoldipine (Sular) | S1748 | 0.6636 | 0.1861 | <0.0001 | | | |
| L-Glutamine | S1749 | 0.8195 | 0.2204 | 0.0028 | | | |
| Gadodiamide (Omniscan) | S1751 | 0.6947 | 0.1333 | <0.0001 | | | |
| Oxybutynin (Ditropan) | S1754 | 0.9915 | 0.2885 | 0.9998 | 1.125 | 0.16 | <0.0001 |
| Enoxacin (Penetrex) | S1756 | 1.045 | 0.2572 | 0.9985 | 1.02 | 0.15 | 0.978 |
| Pitavastatin calcium | S1759 | 0.877 | 0.1976 | 0.2046 | | | |
| (Livalo) | | | | | | | |
| Rifapentine (Priftin) | S1760 | 0.7726 | 0.2357 | <0.0001 | | | |
| Suprofen (Profenal) | S1761 | 0.7755 | 0.2046 | <0.0001 | | | |
| Pyrazinamide (Pyrazinoic | S1762 | 0.8296 | 0.222 | 0.0277 | | | |
| acid amide) | | | | | | | |
| Quetiapine fumarate | S1763 | 0.7917 | 0.2215 | 0.0004 | | | |
| (Seroquel) | | | | | | | |
| Rifampin | S1764 | 1.054 | 0.265 | 0.9922 | 1.049 | 0.18 | 0.1326 |
| (Rifadin, Rimactane) | | | | | | | |
| Beta Carotene | S1767 | 0.8826 | 0.3081 | 0.1851 | 1.075 | 0.15 | 0.0039 |
| Cefditoren pivoxil | S1768 | 0.9366 | 0.2276 | 0.9513 | 1.091 | 0.17 | 0.0001 |
| Sulfadiazine | S1770 | 1.088 | 0.2344 | 0.6499 | 1.17 | 0.22 | <0.0001 |
| Chlorprothixene | S1771 | 0.5751 | 0.1568 | <0.0001 | | | |
| Oxytetracycline | S1773 | 0.8803 | 0.2629 | 0.0216 | | | |
| (Terramycin) | | | | | | | |
| Thioguanine | S1774 | 0.5422 | 0.1342 | <0.0001 | | | |
| Toremifene Citrate | S1776 | 0.7183 | 0.2019 | <0.0001 | | | |
| (Fareston, Acapodene) | | | | | | | |
| Ethionamide | S1777 | 0.7451 | 0.206 | <0.0001 | | | |
| Trifluridine (Viroptic) | S1778 | 0.7468 | 0.2069 | <0.0001 | | | |
| Azacitidine (Vidaza) | S1782 | 0.5322 | 0.1319 | <0.0001 | | | |
| Vidarabine (Vira-A) | S1784 | 0.3742 | 0.1082 | <0.0001 | | | |
| Verteporfin (Visudyne) | S1786 | 0.5249 | 0.0768 | <0.0001 | | | |
| Teniposide (Vumon) | S1787 | 0.4095 | 0.064 | <0.0001 | | | |
| Rifaximin (Xifaxan) | S1790 | 0.3977 | 0.1099 | <0.0001 | | | |
| Bacitracin zinc | S1791 | 0.6175 | 0.1906 | <0.0001 | | | |
| Simvastatin (Zocor) | S1792 | 0.1568 | 0.0091 | <0.0001 | | | |
| Ramipril (Altace) | S1793 | 0.7145 | 0.2146 | <0.0001 | | | |
| Fenofibrate | S1794 | 0.5348 | 0.1352 | <0.0001 | | | |
| (Tricor, Trilipix) | | | | | | | |
| Ranolazine (Ranexa) | S1799 | 0.5015 | 0.2098 | <0.0001 | | | |
| Ranitidine (Zantac) | S1801 | 0.4295 | 0.1104 | <0.0001 | | | |
| Acadesine | S1802 | 0.3637 | 0.1273 | <0.0001 | | | |
| Acetylcholine chloride | S1805 | 1.04 | 0.3146 | 0.9988 | 1.205 | 0.24 | <0.0001 |
| Acipimox | S1806 | 1.03 | 0.3283 | 0.9991 | 1.379 | 0.35 | <0.0001 |
| Acyclovir (Aciclovir) | S1807 | 1.063 | 0.2849 | 0.9596 | 1.225 | 0.25 | <0.0001 |
| Nifedipine (Adalat) | S1808 | 0.9216 | 0.2932 | 0.8421 | 0.9725 | 0.12 | 0.9907 |
| Amiloride hydrochloride | S1811 | 1.018 | 0.2815 | 0.9996 | 1.487 | 0.36 | <0.0001 |
| (Midamor) | | | | | | | |
| Chlorpheniramine Maleate | S1816 | 1.145 | 0.2876 | 0.063 | 1.308 | 0.3 | <0.0001 |
| Clofibrate | S1820 | 1.009 | 0.2631 | 0.9998 | 1.714 | 0.39 | <0.0001 |
| (Atromid-S) | | | | | | | |
| Fenoprofen calcium | S1823 | 0.9731 | 0.2604 | 0.9993 | 1.472 | 0.39 | <0.0001 |
| Erdosteine | S1825 | 0.5728 | 0.1234 | <0.0001 | | | |
| Proparacaine HCl | S1828 | 0.7045 | 0.1725 | <0.0001 | | | |
| Pranlukast | S1829 | 0.9284 | 0.2651 | 0.8852 | 1.394 | 0.31 | <0.0001 |
| Oxfendazole | S1830 | 0.6 | 0.1573 | <0.0001 | | | |
| Carvedilol | S1831 | 0.9878 | 0.27 | 0.9997 | 1.255 | 0.14 | <0.0001 |
| Atracurium besylate | S1832 | 0.6611 | 0.1981 | <0.0001 | | | |
| Butoconazole nitrate | S1833 | 0.6197 | 0.1926 | <0.0001 | | | |
| Azithromycin (Zithromax) | S1835 | 0.8765 | 0.2967 | 0.2593 | | | |
| Flubendazole | S1837 | 1.065 | 0.2995 | 0.9388 | 1.444 | 0.33 | <0.0001 |
| (Flutelmium) | | | | | | | |
| Chloroxine | S1839 | 0.1604 | 0.0115 | <0.0001 | | | |
| Lomustine (CeeNU) | S1840 | 0.9042 | 0.243 | 0.6411 | 1.023 | 0.13 | 0.999 |
| Chenodeoxycholic acid | S1843 | 1.11 | 0.2451 | 0.3577 | 1.45 | 0.3 | <0.0001 |
| Cimetidine (Tagamet) | S1845 | 0.9597 | 0.2624 | 0.9988 | | | |
| Clemastine Fumarate | S1847 | 1.106 | 0.3079 | 0.4609 | 1.237 | 0.28 | <0.0001 |
| Curcumin | S1848 | 0.9059 | 0.2262 | 0.3186 | 0.8098 | 0.06 | <0.0001 |
| Daidzein | S1849 | 0.5394 | 0.148 | <0.0001 | | | |
| Oxibendazole | S1851 | 0.747 | 0.1988 | <0.0001 | | | |
| Penicillamine (Cuprimine) | S1853 | 0.7681 | 0.2191 | <0.0001 | | | |
| Bifonazole | S1854 | 0.6115 | 0.1589 | <0.0001 | | | |
| Pefloxacin mesylate | S1855 | 0.7518 | 0.2129 | <0.0001 | | | |
| Metoprolol tartrate | S1856 | 0.829 | 0.2527 | 0.0007 | | | |
| Etidronate (Didronel) | S1857 | 0.9486 | 0.259 | 0.9482 | 0.992 | 0.22 | 0.9995 |
| Diethylstilbestrol | S1859 | 0.8894 | 0.2419 | 0.1081 | 0.8812 | 0.12 | <0.0001 |
| (Stilbestrol) | | | | | | | |
| Diltiazem HCL (Tiazac) | S1865 | 0.7425 | 0.1992 | <0.0001 | | | |
| Diphenhydramine HCl | S1866 | 0.6149 | 0.1661 | <0.0001 | | | |
| (Benadryl) | | | | | | | |
| Dapoxetine hydrochloride | S1869 | 0.7539 | 0.2411 | <0.0001 | | | |
| (Priligy) | | | | | | | |
| Risedronic acid (Actonel) | S1874 | 0.6565 | 0.1641 | <0.0001 | | | |
| Tranexamic acid | S1875 | 0.7568 | 0.2341 | <0.0001 | | | |
| (Transamin) | | | | | | | |
| Valaciclovir HCl | S1876 | 0.6362 | 0.183 | <0.0001 | | | |
| Ganciclovir | S1878 | 0.5803 | 0.1499 | <0.0001 | | | |
| Roxatidine acetate HCl | S1880 | 0.4245 | 0.1095 | <0.0001 | | | |
| Protionamide | S1881 | 0.6328 | 0.1554 | <0.0001 | | | |
| (Prothionamide) | | | | | | | |
| Idoxuridine | S1883 | 0.7701 | 0.2208 | <0.0001 | | | |
| Sparfloxacin | S1884 | 0.7607 | 0.2471 | <0.0001 | | | |
| Felodipine (Plendil) | S1885 | 1.526 | 0.0994 | <0.0001 | | | |
| Deflazacort (Calcort) | S1888 | 0.5114 | 0.1389 | <0.0001 | | | |
| Nizatidine | S1890 | 0.5754 | 0.1436 | <0.0001 | | | |
| Carbidopa | S1891 | 0.5325 | 0.1309 | <0.0001 | | | |
| Valsartan (Diovan) | S1894 | 0.6351 | 0.1653 | <0.0001 | | | |
| Dipyridamole (Persantine) | S1895 | 0.5847 | 0.1121 | <0.0001 | | | |
| Hydroxyurea (Cytodrox) | S1896 | 0.4236 | 0.1353 | <0.0001 | | | |
| Potassium iodide | S1897 | 0.386 | 0.0896 | <0.0001 | | | |
| Tropisetron | S1898 | 0.5993 | 0.1695 | <0.0001 | | | |
| Nicotinamide (Niacinamide) | S1899 | 0.6872 | 0.1741 | <0.0001 | | | |
| Talc | S1900 | 0.5666 | 0.1471 | <0.0001 | | | |
| Vitamin B12 | S1902 | 0.7029 | 0.1652 | <0.0001 | | | |
| Diclofenac | S1903 | 0.334 | 0.0932 | <0.0001 | | | |
| Avobenzone (Parsol 1789) | S1904 | 0.5309 | 0.0821 | <0.0001 | | | |
| Amlodipine (Norvasc) | S1905 | 1.444 | 0.2953 | <0.0001 | | | |
| Metronidazole (Flagyl) | S1907 | 0.5627 | 0.1326 | <0.0001 | | | |
| Flutamide (Eulexin) | S1908 | 0.6406 | 0.1759 | <0.0001 | | | |
| Fluvastatin sodium (Lescol) | S1909 | 0.5791 | 0.1377 | <0.0001 | | | |
| Tioconazole | S1910 | 0.6333 | 0.147 | <0.0001 | | | |
| Disodium Cromoglycate | S1911 | 0.6798 | 0.1914 | <0.0001 | | | |
| Tropicamide | S1913 | 0.8042 | 0.2076 | <0.0001 | | | |
| Pregnenolone | S1914 | 0.6057 | 0.123 | <0.0001 | | | |
| Sulfamethoxazole | S1915 | 0.8442 | 0.2184 | 0.0008 | | | |
| Sulfisoxazole | S1916 | 0.1947 | 0.0317 | <0.0001 | | | |
| Crystal violet | S1917 | 0.1262 | 0.0071 | <0.0001 | | | |
| Haloperidol (Haldol) | S1920 | 0.6675 | 0.1251 | <0.0001 | | | |
| Phenindione (Rectadione) | S1921 | 0.6802 | 0.1597 | <0.0001 | | | |
| Alibendol | S1928 | 0.7165 | 0.1696 | <0.0001 | | | |
| Irsogladine | S1929 | 0.613 | 0.1713 | <0.0001 | | | |
| Triamcinolone | S1933 | 1.061 | 0.1784 | 0.7792 | 0.9883 | 0.13 | 0.9994 |
| (Aristocort) | | | | | | | |
| Nystatin (Mycostatin) | S1934 | 0.3498 | 0.0744 | <0.0001 | | | |
| Isoniazid (Tubizid) | S1937 | 0.3683 | 0.0755 | <0.0001 | | | |
| Levamisole Hydrochloride | S1939 | 0.8181 | 0.1963 | <0.0001 | | | |
| (Ergamisol) | | | | | | | |
| Levofloxacin (Levaquin) | S1940 | 0.6541 | 0.1826 | <0.0001 | | | |
| Enalapril maleate | S1941 | 0.8307 | 0.2628 | <0.0001 | | | |
| (Vasotec) | | | | | | | |
| Menadione | S1949 | 0.4363 | 0.1316 | <0.0001 | | | |
| Metformin hydrochloride | S1950 | 0.6991 | 0.19 | <0.0001 | | | |
| (Glucophage) | | | | | | | |
| Methoxsalen (Oxsoralen) | S1952 | 0.6397 | 0.1251 | <0.0001 | | | |
| Sulfamethizole (Proklar) | S1957 | 0.4719 | 0.1164 | <0.0001 | | | |
| Sulbactam | S1958 | 0.6491 | 0.1483 | <0.0001 | | | |
| Tolfenamic acid | S1959 | 0.8008 | 0.2379 | <0.0001 | | | |
| Pranoprofen | S1960 | 0.7582 | 0.1688 | <0.0001 | | | |
| Sulphadimethoxine | S1962 | 0.8846 | 0.2175 | 0.0054 | | | |
| Rimantadine (Flumadine) | S1964 | 0.3987 | 0.0875 | <0.0001 | | | |
| Primidone (Mysoline) | S1965 | 0.8725 | 0.1931 | 0.0018 | | | |
| Nefiracetam (Translon) | S1969 | 0.6824 | 0.1485 | <0.0001 | | | |

### Animals

Female SKH1 hairless mice, backcrossed 6 generations with C57BL/6J mice, aged 6-12 weeks were used in this study for in vitro assay development and drug discovery. Female C57BL/6J mice age 6-8 weeks were used for in vivo validation studies. Only female mice were used due to known sex differences in rodent salivary glands, with female glands more accurately emulating human salivary gland structure and function (Pinkstaff, C. A et al., European J Morphology 1998, 36 Suppl, 31-34; Maruyama, C. L et al., Oral Dis 2019, 25, 403-415). Animals were maintained on a 12 hr light/dark cycle and group-housed with food and water available ad libitum. All procedures were approved and conducted in accordance with the University Committee on Animal Resources at the University of Rochester Medical Center (UCAR #2010-24E, UCAR-2008-016E).

### Microbubble (MB) Array Fabrication

Microbubble (MB) arrays were fabricated in poly(dimethyl)siloxane (PDMS) using gas expansion molding as previously described (Song, Y et al., Commun Biol 2021, 4; Giang, U.-B. T et al., Biomed Microdevices 2014, 16, 55-67; Giang, U.-B. T et al., Lab Chip 2007, 7, 1660-1662). PDMS (Dow Corning Sylgard 184) was mixed in a 10:1 ration of base to curing agent and poured over a silicon wafer template consisting of deep etched cylindrical pits with a 200 μm diameter, spaced 600 μm apart. The PDMS was cured at 100 °C for 2 hrs before peeling off the template, resulting in an array of spherical cavities with 200 μm openings and a diameter of ~350 μm. Circular chips with a 0.7 cm diameter (48 well) or 0.5 diameter (96 well) were punched form the PDMS cast and glued into well plates using a 5:1 ratio of PDMS cured at 60 °C for 8 hours. The MB arrays were primed in a desktop vacuum chamber with 70% ethanol to facilitate air removal from the MBs and replacement with fluid. Ethanol was exchanged for PBS and incubated overnight prior to cell seeding. Alternate MB arrays were obtained from commercial sources (MBA™ microbubble array, Nidus MB Technologies).

### Cell Isolation

Mice were euthanized and the submandibular glands (SMG) were removed and chopped with a razor blade for 5 min. The tissue was then incubated in Hank's buffered salt solution (HBSS) containing 15 mM HEPES, 50 U/mL collagenase type II (Thermo Fischer 17101015), and 100 U/mL hyaluronidase (Sigma Aldrich H3506) at 37 °C for 30 min. Cells were centrifuged, then resuspended in HBSS with 15 mM HEPES and passed through 100 μm and 20 μm mesh filters to isolate clusters between 20-100 μm. The digestion protocol produces cell cluster sizes evenly distributed between 20 to 100 μm (Song, Y et al., Advanced Healthcare Materials). The isolated clusters were combined with hydrogel precursor solution and seeded in MB array-based chips as described below.

### MB-Hydrogel Encapsulation of Salivary Gland Cells

Isolated submandibular gland cell clusters (20-100 μm) were encapsulated with poly(ethylene glycol) (PEG) hydrogels within MB arrays (FIG. 1) as previously described (Song, Y et al., Commun Biol 2021, 4). Briefly, the cells (FIG. 1A) were resuspended in hydrogel precursor (FIG. 1B) solution containing 2 mM norbornene-functionalized 4-arm 20 kDa PEG-amine macromers, 4 mM of the dicysteine functionalized MMP degradable peptide (GKKCGPQGIWGQCKKG, SEQ ID NO:1), 0.05 wt % of the photoinitiator lithium phenyl-2,4,6-trimethylbenzoylphosphinate (LAP), and 0.1 mg/mL laminin in PBS (Shubin, A D et al., Tissue Eng: PartA 2015, 21, 1733-1751; Shubin, A D et al., Acta Biomater 2017, 50, 437-449; Song, Y et al., Commun Biol 2021, 4; Fairbanks, B. D et al., Biomaterials 2009, 30, 6702-6707; Shubin, A. D et al., Cell Tissue Res 2020, 380, 487-497). The cell/gel precursor solution (25 μL for 48 well; 20 μL for 96 well) was pipetted onto the MB chips and incubated for 30 min, pipetting every 10 min to redisperse cells that had settled onto the surface of the chip. The hydrogels were polymerized in situ using a Hand-Foot 100 A broad spectrum UV light (UVA: 5 mW/cm²; UVB: −0.4 mW/cm²) with a UVC filter for 1.5 min and cultured with media (0.5 mL for 48 well; 150 μL for 96 well), with media changes every 2 days. Culture medium consisted of Dulbecco's Modified Eagle medium (DMEM):Ham's F-12 Nutrient Mixture (1:1) supplemented with 100 U/mL penicillin and 100 μg/mL streptomycin, 2 mM glutamine, 0.5×N2 supplement, 2.6 ng/mL insulin, 2 nM dexamethasone, 20 ng/mL epidermal growth factor (EGF), and 20 ng/mL basic fibroblast growth factor (bFGF).

### Glutathione Assay

A glutathione assay was developed for in-chip measurements by adapting the Cellular Glutathione Detection Assay Kit (Cell Signaling Technology #13859). The monochlorobimane (MCB) reagent was prepared by reconstitution in DMSO per manufacturer directions. For 96 well plates, 10 μL of the prepared reagent (1:50 ratio of MCB to Tris assay buffer, per manufacturer instructions) was added to wells containing 100 μL of culture media and incubated for 30 min at 37 °C, washed with PBS, and imaged using an Olympus IX70 microscope with a DAPI filter (Excitation: 358 nm; Emission: 461 nm).

### Cellular Senescence Assay

A cellular senescence assay for the chips was developed by adapting the Cellular Senescence Detection Kit—SPiDER-βGal (Dojindo Molecular Technologies, Inc. SG04). Bafilomycin A1 and SPiDER-βGal stock solutions were prepared in DMSO per manufacturer directions. The assay was performed by first incubating the chips with bafilomycin A1 (1:1000 dilution in media) for 1 hr at 37 °C. The solution was removed and replaced with 30 μL of media containing Bafilomycin A1 (1:1000 dilution) and SPiDER-PGal (1:500 dilution) and incubated for 45 min at 37 °C. Chips were washed twice with media and imaged using a fluorescence microscope with a Texas Red filter (Excitation: 580 nm; Emission: 604 nm).

### Drug Mechanism Meta-Analysis

Drug interaction data was examined using PubChem BioAssays results for the hits discovered in the Selleck Chemicals drug library screen described below (Kim, S. et al. Nucleic Acids Res 49, D1388-d1395 (2021); Kim, S. et al. Nucleic Acids Res 44, D1202-1213 (2016); Wang, Y. et al. Nucleic Acids Res 38, D255-266 (2010)). Pathway analysis was performed using the Drug Set Enrichment Analysis (DSEA) tool to identify pathways with gene expression patterns significantly impacted by treatment with the drug hits (p<0.05) (Napolitano, F. et al., Bioinformatics 32, 235-241 (2016)).

### Drug Treatment and Irradiation

For radioprotection experiments, SGm were cultured in MB-hydrogel chips for 4 days, then drugs were added to the chips 30 min prior to irradiation and washed out with media 30 min post-radiation treatment. A dose of 15 Gy ionizing radiation was delivered using a JL Shepherd ¹³⁷Cs irradiator. Drug treatment scheme and radiation dose were established previously (Song, Y et al., Commun Biol 2021, 4). The glutathione and senescence assays were performed at 4- and 5-days post-radiation. For assay validation experiments, at least 3 chips (N=3) were used for each drug, corresponding to >100 MBs (n>100); these values are listed in the figure description for each experiment. Mean, standard deviation, and statistics were calculated based on the number of MBs (n).

For screening of the Selleck Chemicals compound library, the same treatment scheme was used, with drugs administered at 100 μM. One MB chip (N=1) was used per compound, with ~40-50 MBs per chip (n=50-60); statistics were calculated using the number of MBs (n) and compared to 0 Gy controls. Compounds were first screened using the glutathione assay; drugs that exhibited statistically nonsignificant differences compared to the 0 Gy control (hits) with the glutathione assay were then tested with the senescence assay to discover double hits. The glutathione assay was selected as the first screen because it had higher signal-to-noise ratio, more rapid throughput (30 mins versus 2 hrs for completion of the senescent assay), lower assay kit cost, and a higher shelf-life after kit reconstitution.

### Image Quantification and Statistical Analysis

For both the glutathione and cellular senescence assays, images were quantified in ImageJ. Regions of interest (ROIs) were created by thresholding the fluorescence signal (localized to the SGm) and mean intensity of each ROI was measured. Data was graphed and statistical analyses (ANOVA with Tukey's post-hoc test) were performed using GraphPad Prism 9. Schematic diagrams were created using Biorender (biorender.com) under institutional site license, agreement number VP25LG6DJ3.

### Retroductal Injection and Irradiation

Retroductal injection delivery of drugs to the murine submandibular gland has been described in detail (Varghese, J. J. et al. J Vis Exp (2018); Varghese, J. J. et al. J Dent Res 97, 1252-1259 (2018)). Briefly, 6-10 week old female C57Bl/6 J mice were anesthetized by intraperitoneal injection of sterile saline solution of 100 mg/kg ketamine and 10 mg/kg xylazine. Maxillary incisors were secured over a metal beam, while an elastic band provided tension from behind the mandibular incisors. The mouth was widened using a custom steel retractor to apply pressure to the buccal mucosa and the tongue was retracted and cotton placed in the oral cavity. The wire inset of a 32 G intracranial catheter was cut at 450 to create a bevel. The beveled wire created a shallow puncture in the left salivary papilla. A beveled catheter section containing the wire insert for support was gently inserted into the puncture site. The catheter was removed, and 1 mg/kg atropine was administered by intraperitoneal injection. After 10 min, the needle of a Hamilton syringe, loaded with vehicle or drug solution, was inserted into the catheter, and the catheter was inserted into the orifice produced in the papilla. Drug solutions were injected by hand at 10 μl/min at a volume of 1 μl/g of body weight. Following injection, the pressure was maintained on the syringe for 1 min to ensure material retention before removal of the catheter. The cotton and retractor were removed from the oral cavity, and the elastic band and metal beam were released from the incisors (Kim, A. et al. Antimicrob Agents Chemother 52, 2497-2502 (2008)). The known radioprotectant, WR-1065 (50 mg/kg, saline) was used as a control for radioprotection. Phenylbutazone, enoxacin, and doripenem hydrate were treated at 50, 0.3, and 26 mg/kg (N=4-6 per group). Drug doses were chosen based on solubility and published doses for in vivo studies (Kari, F. et al. Jpn J Cancer Res 86, 252-263 (1995); Rocha, A. L. et al. Sci Adv 6 (2020)). Saline was used as the vehicle for all compounds, except phenylbutazone, which required corn oil due to poor aqueous solubility.

Mice injected with vehicle controls (saline or corn oil) or drug were treated with 0 Gy or 15 Gy within 15-30 min of injection, the submandibular glands were irradiated as described previously (Varghese, J. J. et al. J Vis Exp (2018); Arany, S et al. Mol Ther 21, 1182-1194 (2013); Varghese, J. J et al. J Dent Res 97, 1252-1259 (2018)). The head and neck region was positioned over the slit of a custom collimator, which allowed body shielding. Mouse submandibular glands were exposed to a single dose of 15 Gy gamma radiation delivered by a ¹³⁷Cs radiation source. Prior studies demonstrate that this single dose causes xerostomia in mice equivalent to fractionated dosing that recapitulates human sequalae of salivary gland radiation damage (Song, Y. et al. Commun Biol 4, 361 (2021); Cotrim, A. P. et al Clin Cancer Res 13, 4928-4933 (2007)). Animals were allowed to recover for 48 hours to measure persistent long-lived DNA damage, after which, the submandibular glands were harvested, fixed, sectioned, and analyzed using immunohistochemistry.

### Immunohistochemical Analysis

Submandibular glands and SGm were isolated and fixed in 4% paraformaldehyde overnight at 4 °C. Tissues were paraffin-embedded and then cut into 5 m sections. Slides were treated with HIER buffer (10 mM sodium citrate, 0.05% Tween-20, pH 6.0) for antigen retrieval in a pressure cooker for 10 minutes then sections were blocked in CAS-block histochemical reagent (Thermo Fisher Scientific, 008120). Permeabilization was performed with 0.5% Triton X-100 in PBS for 5 minutes. Immunostaining was performed overnight (at 4 °C) with primary antibody for 7H2AX (EMD Millipore, 05-636). Alexa-Fluor 594-conjugated donkey anti-mouse IgG was diluted 1:500 (Invitrogen, A21203) as secondary antibody and applied on sections for 1 hour at room temperature. Following a PBS rinse, 10 g/ml DAPI (Invitrogen, Carlsbad, CA) in PBS was applied to sections for 5 minutes. Sections were washed thrice in PBS for 5 minutes and the slides were mounted using Immu-Mount mounting medium (Thermo). Microscopic images were acquired using a Leica TCS SP5 confocal microscope with a 100× oil immersion objective and Argon laser. Analysis of images was performed in ImageJ.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of preventing radiation damage in a subject or patient in need thereof comprising administering an effective amount of a composition comprising one or more compound selected from the group consisting of: glipizide, etidronate, meropenem, oxcarbazepine, doripenem, phenylbutazone, enoxacin, cilostazol, triamcinolone, didanosine, eplerenone, prazosin HCl, indomethacin, diethylstilbestrol, melatonin, emtricitabine, albendazole, rifampin, nifedipine, rifabutin, calcitriol, lomustine, adefovir dipivoxil, curcumin, gemcitabine, analogs, derivatives, and prodrugs thereof, and pharmaceutically acceptable salts and hydrates thereof.

2. The method of claim 1, wherein the composition comprises one or more selected from the group consisting of glipizide, meropenem, oxcarbazepine, doripenem, phenylbutazone, enoxacin, cilostazol, triamcinolone, didanosine, prazosin HCl, indomethacin, diethylstibestrol, emtricitabine, analogs, derivatives, and prodrugs thereof, and pharmaceutically acceptable salts and hydrates thereof.

3. The method of claim 1, wherein the composition comprises one or more selected from the group consisting of phenylbutazone, meropenem, diethylstilbestrol, prazosin HCl, enoxacin, glipizide, doripenem hydrate, analogs, derivatives, and prodrugs thereof, and pharmaceutically acceptable salts and hydrates thereof.

4. The method of claim 1, wherein the one or more compound is selected from the group consisting of meropenem, diethylstibestrol, glipizide, doripenem hydrate, analogs, derivatives, and prodrugs thereof, and pharmaceutically acceptable salts and hydrates thereof.

5. The method of claim 1, wherein the radiation damage is prevented in the salivary gland of the subject or patient.

6. The method of claim 1, wherein the composition is administered prior to radiation therapy exposure.

7. The method of claim 1, further comprising the step of eliminating the composition from the patient's or subject's body after radiation therapy exposure.

8. The method of claim 1, wherein the composition is administered during radiation therapy exposure.

9. The method of claim 1, wherein the composition is administered prior to and during radiation therapy exposure.

10. The method of claim 1, wherein the composition is administered after radiation therapy exposure.

11. The method of claim 1, further comprising administration of a second radioprotective drug.

12. The method of claim 1, further comprising administering an anesthetic.

13. The method of claim 1, further comprising administering a targeting agent.

14. The method of claim 1, wherein the subject or patient is undergoing radiation therapy.

15. The method of claim 14, wherein the radiation therapy is for treating cancer.

16. The method of claim 15, wherein the cancer is selected from the group consisting of anal cancer, bladder cancer, bone cancer, brain cancer, breast cancer, cervical cancer, chondrosarcoma, chordoma, colon cancer, esophageal cancer, Ewing's sarcoma, head and neck cancer, leukemia, liver cancer, lung cancer, lymphoma, meningioma, non-Hodgkin's lymphoma, osteosarcoma, pancreatic cancer, paranasal sinus cancer, prostate cancer, rectal cancer, skin cancer, soft tissue sarcomas, spinal cord tumors, spine tumors, stomach cancer, uterine cancer, vaginal cancer, and vulval cancer.

17. The method of claim 16, wherein the cancer is head and neck cancer.

18. The method of claim 14, wherein the radiation therapy is for acoustic neuroma, arteriovenous malformations, Graves ophthalmopathy, keloids, orbital pseudotumor, trigeminal neuralgia, and a non-cancerous tumor.
